(19) United States
(12) Patent Application Publication
(10) Pub. No.: US 2023/0381138 A1
(43) Pub. Date: Nov. 30, 2023

(54) LASOFOXIFENE COMBINATION TREATMENT OF ER+ BREAST CANCER THAT HAS PROGRESSED ON A CDK4/6 INHIBITOR

(71) Applicants: Sermonix Pharmaceuticals, Inc., Columbus, OH (US); ELI LILLY AND COMPANY, Indianapolis, IN (US)

(72) Inventors: David J. PORTMAN, Aventura, FL (US); Paul PLOURDE, Philadelphia, PA (US); Simon JENKINS, Philadelphia, PA (US)

(21) Appl. No.: 18/323,674

(22) Filed: May 25, 2023

Related U.S. Application Data
(60) Provisional application No. 63/345,843, filed on May 25, 2022.
(60) Provisional application No. 63/411,633, filed on Sep. 30, 2022.
(60) Provisional application No. 63/426,737, filed on Nov. 19, 2022.
(60) Provisional application No. 63/430,194, filed on Dec. 5, 2022.
(60) Provisional application No. 63/446,760, filed on Feb. 17, 2023.

(51) Int. Cl. A61K 31/40; A61K 31/506; C12Q 1/6886; A61P 35/00

(52) U.S. Cl. CPC A61K 31/40; A61K 31/506; C12Q 1/6886; A61P 35/00; C12Q 2600/158; C12Q 2600/154; C12Q 2600/106

(57) ABSTRACT

Methods for reducing the progression of ER+ breast cancer in a patient are provided, the methods comprising administering to the patient an effective amount of lasofoxifene or a pharmaceutically acceptable salt thereof and an effective amount of a CDK4/6 inhibitor (CDK4/6i), wherein the breast cancer: (i) is estrogen receptor positive (ER⁺); (ii) has at least one gain of function missense mutation within the ligand binding domain (LBD) of the Estrogen Receptor 1 (ESR1) gene; and (iii) has progressed during prior CDK4/6 inhibitor therapy and/or has an oncogenic mutation in a gene other than ESR1.

### 1. CROSS-REFERENCES TO RELATED APPLICATIONS

This application claims benefit of and priority to U.S. Provisional Application No. 63/345,843, filed May 25, 2022; U.S. Provisional Application No. 63/411,633, filed Sep. 30, 2022; U.S. Provisional Application No. 63/426,737, filed Nov. 19, 2022; U.S. Provisional Application No. 63/430,194, filed Dec. 5, 2022; and U.S. Provisional Application No. 63/446,760, filed Feb. 17, 2023, the disclosure of each of which is incorporated herein by reference in its entirety.

### 2. BACKGROUND OF THE INVENTION

Estrogen receptor positive (ER⁺) breast cancers express estrogen receptor a (ERα), which is encoded by the ESR1 gene. Approximately 70% of breast cancers are ER⁺ and are, therefore, treated with agents that deplete circulating estrogen levels or that block estrogen signaling in the cancer cell (collectively, endocrine therapy). Endocrine therapy has led to significant improvement in outcome of women with ER⁺ breast cancer. However, the effectiveness of endocrine therapy is limited by intrinsic and, importantly, acquired endocrine resistance. In response to the selective pressure imposed by endocrine therapies, in particular by aromatase inhibitors (AI), ER⁺ tumors evolve various escape mechanisms. Among these is acquisition of gain-of-function mutations in the ESR1 gene that alter the ligand binding domain of the ERα receptor, rendering the receptor constitutively active at low levels, or in the absence, of estrogen. Despite the benefits of endocrine therapy, the majority of ER⁺ tumors will eventually acquire resistance and progress.

Lasofoxifene, a third generation selective estrogen receptor modulator (SERM), has been shown to reduce the risk of invasive ER⁺ breast cancer in women with wild type estrogen receptors: i.e., post-menopausal women with no history of breast cancer being treated for osteoporosis. LaCroix et al., J. Natl. Cancer Inst. 102:1706-1715 (2010). Lasofoxifene was later shown to retain the ability to inhibit progression of ER⁺ cancers that have developed gain-of-function mutations in the ligand binding domain of the ERα receptor (ESR1 gene). U.S. Pat. Nos. 10,258,605 and 10,905,659; WO 2019/199891; Lainé et al., Breast Cancer Res. 23(1):54 (2021). The efficacy of lasofoxifene as a single agent in the treatment of premenopausal and postmenopausal women with locally advanced or metastatic ER⁺ breast cancers that have acquired ESR1 gain-of-function mutations is currently being confirmed in a phase 2 clinical trial, NCT03781063 (the ELAINE trial).

Over the last decade, a new class of drugs, cyclin dependent kinase 4/6 inhibitors (CDK4/6i), have become commercially available for the treatment of women with (ER⁺) breast cancer. Three CDK4/6i have been approved in a number of countries around the world, including the United States, for treatment of ER⁺ cancers in combination with endocrine therapy: palbociclib (IBRANCE, Pfizer), ribociclib (KISQALI, Novartis) and abemaciclib (VERZENIO, Eli Lilly). However, ER⁺ tumors have been shown to develop resistance to CDK4/6i and ultimately progress.

There remains a need for new therapies that are effective to treat ER⁺ tumors harboring mutations in ESR1 and that have developed resistance to, and thus progressed on, endocrine therapies and CDK4/6 inhibitors.

### 3. SUMMARY OF THE INVENTION

The ongoing ELAINE 2 clinical trial is an open-label, multicenter, study evaluating the efficacy, safety and tolerability of the combination of the third generation SERM, lasofoxifene, and the CDK4/6 inhibitor, abemaciclib, for the treatment of pre-menopausal and post-menopausal women who have locally advanced or metastatic ER⁺/HER2⁻ breast cancer with an ESR1 mutation and disease progression on first, second, or third lines of hormonal treatment for metastatic disease. To be eligible for enrollment, progression may have occurred after up to three of the following treatments for metastatic breast cancer: an aromatase inhibitor (AI) and/or fulvestrant, either as monotherapy or in combination with any commercially available CDK4/6i; and/or the combination of fulvestrant and alpelisib; and/or tamoxifen; and/or the combination of exemestane/everolimus; and up to one line of chemotherapy in the metastatic setting (48%). A majority of the patients enrolled in the trial (28/29) had progressed on prior CDK4/6 inhibitor combination therapy. We have now discovered that the combination of lasofoxifene with the CDK4/6 inhibitor (CDK4/6i) abemaciclib is well tolerated and demonstrates robust and meaningful efficacy in such patients, reducing progression of breast cancer in women with locally advanced or metastatic ER⁺ breast cancer harboring an ESR1 gain-of-function mutation who had progressed on prior CDK4/6i therapies.

In addition, we have now discovered that the combination of lasofoxifene with the CDK4/6 inhibitor (CDK4/6i) abemaciclib reduces progression of breast cancer in women with locally advanced or metastatic ER+ breast cancer harboring an ESR1 gain-of-function mutation and an oncogenic mutation in one or more genes other than the ESR1 gene.

Accordingly, in a first aspect, provided herein is a method of reducing the progression of breast cancer in a patient, comprising:
administering to the patient an effective amount of lasofoxifene or a pharmaceutically acceptable salt thereof and an effective amount of a CDK4/6 inhibitor (CDK4/6i),
wherein the breast cancer:
   (i) is estrogen receptor positive (ER⁺);
   (ii) has at least one gain of function missense mutation within the ligand binding domain (LBD) of the Estrogen Receptor 1 (ESR1) gene; and
   (iii) has progressed during prior CDK4/6 inhibitor therapy.

In one aspect, provided herein is a method of reducing the progression of breast cancer in a patient, comprising:
administering to the patient an effective amount of lasofoxifene or a pharmaceutically acceptable salt thereof and an effective amount of a CDK4/6 inhibitor (CDK4/6i),
wherein the breast cancer:
   (i) is estrogen receptor positive (ER⁺);
   (ii) has at least one gain of function missense mutation within the ligand binding domain (LBD) of the Estrogen Receptor 1 (ESR1) gene; and
   (iii) has an oncogenic mutation in one or more genes other than the ESR1 gene.

In one aspect, provided herein is a method of reducing the progression of breast cancer in a patient, comprising:
administering to the patient an effective amount of lasofoxifene or a pharmaceutically acceptable salt thereof and an effective amount of a CDK4/6 inhibitor (CDK4/6i),
wherein the breast cancer:
   (i) is estrogen receptor positive (ER⁺);
   (ii) has at least one gain of function missense mutation within the ligand binding domain (LBD) of the Estrogen Receptor 1 (ESR1) gene; and
   (iii) has increased expression of one or more genes other than the ESR1 gene.

In one aspect, provided herein is a method of monitoring a patient on a breast cancer treatment, comprising:
(a) determining quantitative measures of the mutant allele frequency (MAF) of circulating tumor DNA (ctDNA) of at least one gain of function missense mutation within the ligand binding domain (LBD) of the Estrogen Receptor 1 (ESR1) gene (ESR1 ctDNA) in a biological sample of the patient, wherein the quantitative measures are performed over a period of time at determined intervals; and
(b) determining a positive predictive value (PPV) for clinical benefit with stable disease of the cancer treatment,
   wherein the PPV indicates responsiveness to the cancer treatment,
   wherein the cancer treatment comprises an effective amount of lasofoxifene or a pharmaceutically acceptable salt thereof and an effective amount of a CDK4/6 inhibitor (CDK4/6i).

The method of any preceding embodiment, wherein the ER⁺ breast cancer is HER2⁻.

The method of any preceding embodiment, wherein the ER⁺ breast cancer is locally advanced or metastatic.

The method of any preceding embodiment, wherein lasofoxifene is administered as lasofoxifene tartrate.

The method of any preceding embodiment, wherein lasofoxifene is administered at 5 mg/day per os.

The method of any preceding embodiment, wherein the CDK4/6i administered to the patient is selected from palbociclib, ribociclib, and abemaciclib.

The method of any preceding embodiment, wherein the CDK4/6i administered to the patient is abemaciclib. In certain embodiments, abemaciclib is administered orally at 50 mg to 200 mg BID. In certain embodiments, abemaciclib is administered orally at 100 mg to 200 mg BID. In certain embodiments, abemaciclib is administered orally at 150 mg BID.

The method of any preceding embodiment, wherein the prior administered CDK4/6 inhibitor is selected from palbociclib, ribociclib, and abemaciclib. In certain of these embodiments, the prior administered CDK4/6 inhibitor is abemaciclib.

The method of any preceding embodiment, wherein the cancer has previously been determined to have at least one gain of function missense mutation within the ligand binding domain (LBD) of the Estrogen Receptor 1 (ESR1) gene.

The method of any preceding embodiment, further comprising an earlier step of:
determining that the patient has at least one gain of function missense mutation within the ligand binding domain (LBD) of the Estrogen Receptor 1 (ESR1) gene.

The method of any preceding embodiment, wherein the at least one of gain of function missense mutation is in any one of amino acids D538, Y537, L536, P535, V534, L469, 5463, V392, and E380.

### 4. BRIEF DESCRIPTION OF THE SEVERAL VIEWS OF THE DRAWINGS

These and other features, aspects, and advantages of the present invention will become better understood with regard to the following description, and accompanying drawings, in which:

FIG. 1 is a swimmer plot presenting data on individual patient response to treatment with lasofoxifene and abemaciclib in the ELAINE 2 clinical trial (NCT04432454) on a first interim date.

FIG. 2 illustrates patient maximum tumor response at the same timepoint as FIG. 1 (PD=progressive disease; SD=stable disease; PR=partial remission).

FIG. 3 is a swimmer plot presenting data on patient response to treatment with lasofoxifene and abemaciclib in the ELAINE 2 clinical trial (NCT04432454) at a later interim date than FIGS. 1 and 2, with additional information on each subject's pre-enrollment treatments.

FIG. 4 depicts, for each individual subject enrolled in the ELAINE 2 trial, the duration of the subject's pre-enrollment response on prior second-line and third-line cancer therapies versus on-trial duration of response to the combination of lasofoxifene and abemaciclib at the same timepoint as in FIG. 3.

FIG. 5 presents prevalence of oncogenic mutations in a panel of genes, including the ESR1 gene and other genes known or suspected to contribute to neoplasia in enrolled ELAINE 2 subjects at baseline, and the subject's subsequent achievement of clinical benefit (CB) and the median progression-free survival (mPFS) on treatment with lasofoxifene and abemaciclib in the ELAINE 2 trial, as of the interim date of FIG. 3.

FIG. 6 presents individual MAF kinetics for the most commonly observed mutESR1 variants. Shown are Y537C, Y537N, Y537S, and D538G variants. Variants with low baseline MAF (solid line with diamond ends) utilize the left vertical axis (y-axis) and those with high baseline MAF (broken line with circle ends) utilize the right vertical axis (y-axis). X-axis: timeline from baseline to week 4. BL: baseline; MAF: mutant allele fraction.

FIG. 7 is a swimmer plot presenting data on patient response to treatment with lasofoxifene and abemaciclib in the ELAINE 2 clinical trial (NCT04432454) at an interim date later than FIGS. 1-4. Also shown are additional information on each subject's pre-enrollment treatments.

FIGS. 8A and 8B illustrate exemplary types of copy number variations (CNVs) of various oncogenes, including CCND1, detected in circulating tumor DNA (ctDNA) of subjects in the ELAINE 2 clinical trial (NCT04432454), as of the date of FIG. 5. FIG. 8A shows CNV events detected per gene. FIG. 8B shows copy number distribution per gene for the CNV events shown on the left panel. CNVs were annotated as “focal” or “aneuploidy”, or “amplification” where focal or aneuploidy status was indeterminate.

FIG. 9 presents prevalence of oncogenic mutations in a panel of genes, including the ESR1 gene and other genes known or suspected to contribute to neoplasia in enrolled ELAINE 2 subjects at baseline, and the subject's subsequent achievement of clinical benefit (CB) and the median progression-free survival (mPFS) on treatment with lasofoxifene and abemaciclib in the ELAINE 2 trial, at an interim date later than FIG. 5.

FIGS. 10A-10B are swimmer plots presenting data on patient response to treatment with lasofoxifene and abemaciclib in the ELAINE 2 clinical trial (NCT04432454) at an interim date later than FIGS. 1-4 and 7. FIG. 10B is a duplicate of FIG. 10A including individual patient (albeit de-identified) designation numbers. Also shown are additional information on each subject's pre-enrollment treatments.

### 5. DETAILED DESCRIPTION OF THE INVENTION

The ongoing ELAINE 2 clinical trial is an open-label, multicenter, study evaluating the efficacy, safety and tolerability of the combination of the third generation SERM, lasofoxifene, and the CDK4/6 inhibitor, abemaciclib, for the treatment of premenopausal and postmenopausal women who have locally advanced or metastatic ER⁺/HER2⁻ breast cancer with an ESR1 mutation and who have had disease progression on first, second or third lines of hormonal treatment for metastatic disease. To be eligible for enrollment, progression may have occurred on no more than three of the following treatments for metastatic breast cancer: an aromatase inhibitor (AI) and/or fulvestrant, either as monotherapy or in combination with any commercially available CDK4/6i; and/or the combination of fulvestrant and alpelisib; and/or tamoxifen; and/or the combination of exemestane/everolimus.

All but one (28/29) of the patients enrolled in the trial had progressed on prior CDK4/6 inhibitor combination therapy. We have now discovered that the combination of lasofoxifene with the CDK4/6 inhibitor (CDK4/6i) abemaciclib is well tolerated and demonstrates robust and meaningful efficacy in such patients, reducing progression of breast cancer in women with advanced ER⁺ breast cancer harboring a gain of function mutation in the ligand binding domain of ESR1 and who had progressed on prior CDK4/6i therapies.

In addition, we have now discovered that the combination of lasofoxifene with the CDK4/6 inhibitor (CDK4/6i) abemaciclib reduces progression of breast cancer in women with locally advanced or metastatic ER+ breast cancer harboring an ESR1 gain-of-function mutation and an oncogenic mutation in one or more genes other than the ESR1 gene.

### 5.1. Methods of Treatment

Accordingly, in a first aspect, disclosed herein are methods of reducing (or slowing) the progression of breast cancer in a patient by administering to the patient an effective amount of lasofoxifene or a pharmaceutically acceptable salt thereof and an effective amount of a CDK4/6 inhibitor (CDK4/6i), wherein the breast cancer: (i) is estrogen receptor positive (ER⁺); (ii) has at least one gain-of-function missense mutation within the ligand binding domain (LBD) of the Estrogen Receptor 1 (ESR1) gene; and (iii) has progressed during prior CDK4/6 inhibitor therapy.

In certain embodiments, the patient's breast cancer is human epidermal growth factor receptor 2 negative (HER2⁻). In certain embodiments, the patient's breast cancer is locally advanced. In certain embodiments, the patient's breast cancer is metastatic.

Also disclosed herein are methods of treating breast cancer in a patient by administering to the patient an effective amount of lasofoxifene or a pharmaceutically acceptable salt thereof and an effective amount of a CDK4/6 inhibitor (CDK4/6i), wherein the breast cancer: (i) is estrogen receptor positive (ER⁺); (ii) has at least one gain-of-function missense mutation within the ligand binding domain (LBD) of the Estrogen Receptor 1 (ESR1) gene; and (iii) has an oncogenic mutation in one or more genes other than the ESR1 gene. In certain embodiments, the patient's cancer is human epidermal growth factor receptor 2 negative (HER2⁻). In certain embodiments, the patient's ER⁺ breast cancer is locally advanced. In certain embodiments, the patient's ER⁺ breast cancer is metastatic.

### 5.1.1. Patient with ER⁺ Cancer

In accordance with the provided methods of treatment, the patient has ER⁺ breast cancer. In various embodiments, the patient has been diagnosed with ER⁺ breast cancer by immunohistochemistry (IHC) performed on a sample of the patient's cancer.

In some embodiments, the patient is premenopausal, perimenopausal or postmenopausal. In some embodiments, the patient is premenopausal and has locally advanced or metastatic ER⁺ breast cancer. In some embodiments, the patient is perimenopausal and has locally advanced or metastatic ER⁺ breast cancer. In some embodiments, the patient is postmenopausal and has locally advanced or metastatic ER⁺ breast cancer.

In some embodiments of the provided methods of treatment, the patient's breast cancer is HER2⁻ (ER⁺/HER2⁻). In particular embodiments, the patient has locally advanced or metastatic ER⁺/HER2⁻ breast cancer.

### 5.1.2. Mutations in ESR1 Gene

In accordance with the provided methods of treatment, cells of the patient's cancer have acquired at least one gain-of-function missense mutation within the ligand binding domain (LBD) of the Estrogen Receptor 1 (ESR1) gene.

In some embodiments, the mutation leads to the ligand-independent activity of the estrogen receptor. In some embodiments, the mutation leads to enhanced ligand-stimulated activity of the estrogen receptor. In some embodiments, the mutation leads to resistance to endocrine therapy. In some embodiments, the mutation promotes tumor growth. In some embodiments, the mutation enhances metastatic activity of the cancer. In some embodiments, the mutation enhances further metastatic activity of ER⁺ metastatic breast cancer.

In various embodiments, the mutation arises from a rare and undetectable pre-existing clone. In some embodiments, the mutation is acquired de novo during the course of endocrine therapy treatment. In some embodiments, the mutation is acquired de novo after multiple lines of endocrine therapy treatment. In some embodiments, the mutation is acquired de novo after multiple lines of endocrine therapy treatment of metastatic breast cancer. In various embodiments, the mutant clone expands to become a more dominant clone over the course of successive lines of endocrine therapy.

In some embodiments, the mutation in the ESR1 gene is a missense point mutation. In some embodiments, the mutation in the ESR1 gene is a truncating mutation. In some embodiments, the mutation in the ESR1 gene is a gene amplification. In some embodiments, the mutation in the ESR1 gene is a genomic rearrangement.

In some embodiments, the patient has an ER⁺ breast cancer that has at least one gain-of-function missense mutation within the ligand binding domain (LBD) of the ESR1 gene. In various embodiments, at least one of the mutations is in an amino acid selected from D538, Y537, L536, P535, V534, L469, S463, V392, and E380, wherein the amino acids are numbered according to the ESR1 protein with NCBI accession number NP_000116.2.

In particular embodiments, the mutation increases the stability of the agonist conformation of Helix 12 of the ERα protein. In some of these embodiments, the mutation increases the binding of the estrogen receptor to its co-activators. In some of these embodiments, the mutation leads to hormone independent activity of estrogen receptor. In some of these embodiments, the mutation leads to resistance to tamoxifen, fulvestrant, and/or aromatase inhibitors.

In certain embodiments, the mutation is in amino acid D538. In certain preferred embodiments, the mutation is D538G. In certain embodiments, the ER⁺ breast cancer has at least a D538G mutation and at least one mutation in an amino acid selected from Y537, L536, P535, V534, L469, S463, V392, and/or E380. In certain embodiments, the ER⁺ breast cancer has at least a D538 mutation and at least a Y537 mutation.

In certain embodiments, the mutation is in amino acid Y537. In certain embodiments, the ER⁺ breast cancer has at least one mutation in amino acid Y537 and at least one mutation in an amino acid selected from D538, L536, P535, V534, L469, S463, V392, and/or E380. In some of these embodiments, the mutation is Y537S, Y537N, Y537C, or Y537Q. In certain preferred embodiments, the mutation is Y537S. In certain preferred embodiments, the mutation is Y537C. In certain preferred embodiments, the mutation is Y537N. In certain preferred embodiments, the mutation is Y537Q.

In some embodiments, the mutation is in amino acid L469. In certain embodiments, the ER⁺ breast cancer has at least one mutation in amino acid L469 and at least one mutation in an amino acid selected from D538, L536, Y537, P535, V534, S463, V392, and/or E380. In certain preferred embodiments, the mutation is L469V.

In some embodiments, the mutation is in amino acid L536. In certain embodiments, the ER⁺ breast cancer has at least one mutation in amino acid L536 and at least one mutation in an amino acid selected from D538, Y537, P535, V534, L469, S463, V392, and/or E380. In certain embodiments, the mutation is L536R or L536Q. In certain embodiments, the mutation is L536R. In certain embodiments, the mutation is L536Q. In certain embodiments, the mutation is L536P. In certain embodiments, the mutation is L536H.

In some embodiments, the mutation is in amino acid P535. In certain embodiments, the ER⁺ breast cancer has at least one mutation in amino acid P535 and at least one mutation in an amino acid selected from D538, Y537, L536, V534, L469, S463, V392, and/or E380. In certain embodiments, the mutation is P535H.

In some embodiments, the mutation is in amino acid V534. In certain embodiments, the ER⁺ breast cancer has at least one mutation in amino acid V534 and at least one mutation in an amino acid selected from D538, Y537, L536, P535, L469, S463, V392, and/or E380. In certain embodiments, the mutation is V534E.

In some embodiments, the mutation is in amino acid S463. In certain embodiments, the ER⁺ breast cancer has at least one mutation in amino acid S463 and at least one mutation in an amino acid selected from D538, Y537, L536, P535, V534, L469, V392, and/or E380. In certain embodiments, the mutation is S463P.

In some embodiments, the mutation is in amino acid V392. In certain embodiments, the ER⁺ breast cancer has at least one mutation in amino acid V392 and at least one mutation in an amino acid selected from D538, Y537, L536, P535, V534, L469, S463, and/or E380. In certain embodiments, the mutation is V3921.

In some embodiments, the mutation is in amino acid E380. In certain embodiments, the ER⁺ breast cancer has at least one mutation in amino acid E380 and at least one mutation in an amino acid selected from D538, Y537, L536, P535, V534, L469, S463, and/or S463. In certain embodiments, the mutation is E380Q.

### 5.1.2.1 Detection of the ESR1 Gene Mutations

In various embodiments, the patient's ER⁺ breast cancer has been previously determined to have at least one mutation in the ESR1 gene. Some embodiments of the methods described herein further include the earlier step of detecting the mutations in the ESR1 gene.

In some embodiments, massively parallel next generation sequencing (NGS) is used for detecting the estrogen receptor mutations in the patient's cancer. In certain embodiments, the entire genome is sequenced. In certain embodiments, selected gene panels of cancer-related genes are sequenced. In certain embodiments, all coding exons within a given set of genes are sequenced. In certain embodiments, known “hotspot” regions within a given set of genes are sequenced. However, the inherent error rate of current next generation sequencing techniques is up to 1%, limiting the sensitivity and specificity of detection. In some embodiments, targeted sequencing is used for detecting the presence of the ESR1 mutations. Although targeted sequencing allows deeper sequencing, it is also currently limited by the 1% error rate. In some embodiments, methods with reduced sequencing error rate are used. In a particular embodiment, Safe-Sequencing System (Safe-SeqS) is used, which tags each template molecule to allow for confident identification of rare variants. See Kinde et al., Proceedings of the National Academy of Sciences 108(23): 9530-9535 (2011). In particular embodiments, ultrasensitive Duplex sequencing is used, which independently tags and sequences each of the two strands of a DNA duplex. See Schmitt et al., Proceedings of the National Academy of Sciences 109(36): 14508-14513 (2012). In some embodiments, digital droplet PCR is used, which emulsifies DNA in thousands to millions of droplets to encapsulate single DNA molecules, designed with mutant specific primers. See Vogelstein and Kinzler, Proceedings of the National Academy of Sciences 96(16): 2322-2326 (1999) and Huggett et al., Clinical Chemistry 61(1): 79-88 (2014).

In some embodiments, the detection of the ESR1 mutations occurs along with the initial diagnosis. In some embodiments, the detection of the mutations occurs at the time of evaluating disease progression, relapse, or recurrence. In some embodiments, the detection of the mutations occurs at the time of disease progression. In some embodiments, the detection of the mutations takes place at the time when the disease is stable.

In some embodiments, one or more biologic specimens are obtained from the patient for detection of the mutations. In certain embodiments, the biologic specimen is a tissue specimen. In certain embodiments, the tissue specimen is a tumor biopsy. In certain embodiments, the tissue specimen is a biopsy of metastases. In some other embodiments, the biologic specimen is a body fluid, e.g., obtained from peripheral blood (liquid biopsy). In certain embodiments, the liquid biopsy comprises circulating tumor cells (CTCs). In certain embodiments, the liquid biopsy comprises cell free DNA.

In specific embodiments of the methods provided herein, the ESR1 mutations are monitored by analysis of circulating tumor DNA (ctDNA). In some embodiments, the ctDNA analysis is performed, e.g., intermittently or regularly, throughout the duration of treatment. In some of these embodiments, the ctDNA is extracted from patient blood samples. In certain embodiments, the ctDNA is evaluated by digital PCR analysis of the ESR1 mutations.

In some embodiments, ctDNA analysis is performed by a liquid biopsy assay as a companion diagnostic device to identify patients with breast cancer lacking or having ESR1 mutations. Exemplary liquid biopsy assays are Guardant360® CDx (2021, FDA approved panel or professional services panel [guardant360cdx.com/gene-list/]), Guardant360 Response™ (2021, [ncbi.nlm.nih.gov/gtr/tests/593444/]), and FoundationOne® Liquid CDx (2021, [assets. ctfassets. net/w98cd481qypO/wVEm7VtICYROsT5 C1VbU7/fd055e0476183 a6acd4eae6b 583e3a00/F1LCDx_Technical_Specs_072021.pdf]), each of which is incorporated herein by reference in its entirety.

### 5.1.3. Patient with Oncogenic Mutations Other Than ESR1 Mutations

In one aspect, disclosed herein are methods of reducing (or slowing) the progression of breast cancer in a patient by administering to the patient an effective amount of lasofoxifene or a pharmaceutically acceptable salt thereof and an effective amount of a CDK4/6 inhibitor (CDK4/6i), wherein the breast cancer: (i) is estrogen receptor positive (ER⁺); (ii) has at least one gain of function missense mutation within the ligand binding domain (LBD) of the Estrogen Receptor 1 (ESR1) gene; and (iii) has an oncogenic mutation in one or more genes other than the ESR1 gene. In some embodiments, the oncogenic mutation is detected in the circulating tumor DNA (ctDNA) in a biological sample obtained from the patient. In some embodiments, the biological sample is blood, plasma, serum, or bodily fluids (such as saliva, tear, seminal fluid, cervical fluid, urine, cerebrospinal fluid, peritoneal fluid, pleural fluid, amniotic fluid, or extracellular fluid). In some embodiments, the biological sample is plasma.

In certain embodiments, the patient's ER⁺ breast cancer is locally advanced. In certain embodiments, the patient's ER⁺ breast cancer is metastatic.

In some embodiments, at least one of the one or more genes with an oncogenic mutation is selected from HNF1A, TERT, GATA3, CDK12, MAPK3, GNAS, RAF1, RHEB, NTRK3, TP53, PIK3CA, APC, ATM, MET, CCND1, EGFR, ROS1, BRCA2, STK11, FGFR1, ARID1A, CCNE1, AR, ALK, ERBB2, KIT, CDK4, SMAD4, NOTCH1, RB1, BRAF, PTEN, AKT1, CDH1, BRCA1, MYC, CDKN2A, IDH2, MTOR, or PDGFRA. In some embodiments, each of the one or more genes with an oncogenic mutation is selected from HNF1A, TERT, GATA3, CDK12, MAPK3, GNAS, RAF1, RHEB, NTRK3, TP53, PIK3CA, APC, ATM, MET, CCND1, EGFR, ROS1, BRCA2, STK11, FGFR1, ARID1A, CCNE1, AR, ALK, ERBB2, KIT, CDK4, SMAD4, NOTCH1, RB1, BRAF, PTEN, AKT1, CDH1, BRCA1, MYC, CDKN2A, IDH2, MTOR, or PDGFRA.

In certain embodiments, treatment with lasofoxifene in combination with a CDK4/6i (e.g., palbociclib, ribociclib, abemaciclib) in patients having the at least one or more oncogenic mutations provides a median progression-free survival (mPFS) of at least 4 weeks, 8 weeks, 12 weeks, 16 weeks, 20 weeks, 24 weeks, 28 weeks, 32 weeks, 36 weeks, 40 weeks, 44 weeks, 48 weeks, 52 weeks, 56 weeks, 60 weeks, 64 weeks, 68 weeks, 72 weeks, 76 weeks, 80 weeks, 84 weeks, 88 weeks, 92 weeks, 96 weeks, 100 weeks, or longer. In some embodiments, treatment with lasofoxifene in combination with abemaciclib provides the patient a mPFS of at least 24 weeks. In some embodiments, the individual patient has one or more genes with an oncogenic mutation detected in the patient's ctDNA or cancer and the one or more genes is selected from HNF1A, TERT, GATA3, CDK12, MAPK3, TP53, PIK3CA, APC, ATM, MET, CCND1, EGFR, ROS1, STK11, FGFR1, ARID1A, CCNE1, AR, ALK, ERBB2, KIT, CDK4, SMAD4, NOTCH1, RB1, BRAF, RAF1, PTEN, AKT1, CDH1, BRCA1, MYC, CDKN2A, or PDGFRA.

In certain embodiments, treatment with lasofoxifene in combination with a CDK4/6i (e.g., palbociclib, ribociclib, abemaciclib) in patients having the at least one or more oncogenic mutations provides clinical benefit (CB; defined as stable disease ≥24 weeks, or confirmed partial or complete response) in at least 10%, 15%, 20%, 25%, 30%, 35%, 40%, 45%, 50%, 55%, 60%, 65%, 70%, 75%, 80%, 85%, 90%, 95%, or 100% of the patient population. In some embodiments, treatment with lasofoxifene in combination with abemaciclib provides CB (defined as stable disease ≥24 weeks, or confirmed partial or complete response) to about 90%, about 95%, or about 100% of the patient population. In some embodiments, the individual patient has one or more genes with an oncogenic mutation detected in the patient's ctDNA or cancer and the one or more genes is selected from CCND1, FGFR1, CCNE1, AR, ALK, MAPK3, KIT, SMAD4, NOTCH1, RB1, BRAF, RAF1, PTEN, AKT1, CDH1, BRCA1, MYC, CDKN2A, or PDGFRA.

In some embodiments, the individual patient has one or more genes with an oncogenic mutation detected in the patient's ctDNA or cancer and the one or more genes are selected from TP53, PIK3CA, CCND1, ARID1A, FGFR1, CCNE1 and ERBB2, and combinations thereof. In some embodiments, one or more oncogenic mutations in each of the genes TP53, PIK3CA, CCND1, ARID1A, FGFR1, CCNE1 and ERBB2, is detected in the individual patient's ctDNA or cancer. In some embodiments, the oncogenic mutations are selected from single nucleotide variants (SNVs), insertions and deletions (indels), copy number variations (CNV) (focal, aneuploidy, amplification), fusions, and combinations thereof.

In some embodiments, the one or more genes with an oncogenic mutation have at least 1%, 5%, 10%, 15%, 20%, 25%, 30%, 35%, 40%, 45%, 50%, 55%, 60%, 65%, 70%, 75%, 80%, 85%, 90%, 95%, or 100% prevalence in the patient population who respond positively to the treatment with lasofoxifene in combination with a CDK4/6i (e.g., palbociclib, ribociclib, abemaciclib) (e.g., achieve CB, defined as stable disease ≥24 weeks, or confirmed partial or complete response). In some embodiments, the one or more genes with an oncogenic mutation have at least 17% prevalence in the patient population who respond positively (e.g., achieve CB) to the treatment with lasofoxifene in combination with a CDK4/6i (e.g., palbociclib, ribociclib, abemaciclib). In some embodiments, the individual patient has one or more genes with an oncogenic mutation detected in the patient's ctDNA or cancer and the one or more genes is selected from HNF1A, TERT, GATA3, CDK12, MAPK3, TP53, PIK3CA, APC, ATM, MET, CCND1, EGFR, ROS1, BRCA2, STK11, FGFR1, ARID1A, CCNE1, AR, ALK, ERBB2, KIT, BRAF, or CDK4.

In some embodiments, the one or more genes with an oncogenic mutation is detected in the patient's ctDNA or cancer and the one or more genes is selected from HNF1A, TERT, GATA3, CDK12, MAPK3, GNAS, RAF1, RHEB, or NTRK3. In some embodiments, the ctDNA or cancer does not have an oncogenic mutation in one or more genes selected from GNAS, RHEB, NTRK3, IDH2, or mTOR. In some embodiments, the ctDNA or cancer does not have an oncogenic mutation in IDH2 or mTOR.

In one aspect, disclosed herein are methods of reducing (or slowing) the progression of breast cancer in a patient by administering to the patient an effective amount of lasofoxifene or a pharmaceutically acceptable salt thereof and an effective amount of a CDK4/6 inhibitor (CDK4/6i), wherein the breast cancer: (i) is estrogen receptor positive (ER⁺); (ii) has at least one gain of function missense mutation within the ligand binding domain (LBD) of the Estrogen Receptor 1 (ESR1) gene; and (iii) has increased expression of one or more genes other than the ESR1 gene as compared to the expression in non-cancerous breast cells in the subject or the expression level in a subject who does not have breast cancer. In certain embodiments, the patient's ER⁺ breast cancer is locally advanced. In certain embodiments, the patient's ER⁺ breast cancer is metastatic. In some embodiments, at least one of the one or more genes with an increased level of expression detected in the patient's ctDNA or cancer is selected from ABL1, AKT1, AKT2, ALK, APC, AR, ARID1A, ASXL1, ATM, AURKA, BAP, BAP1, BCL2L11, BCR, BRAF, BRCA1, BRCA2, CCND1, CCND2, CCND3, CCNE1, CDH1, CDK4, CDK6, CDK8, CDKN1A, CDKN1B, CDKN2A, CDKN2B, CEBPA, CTNNB1, DDR2, DNMT3A, E2F3, EGFR, EML4, EPHB2, ERBB2, ERBB3, ESR1, EWSR1, FBXW7, FGF4, FGFR1, FGFR2, FGFR3, FLT3, FRS2, HIF1A, HRAS, IDH1, IDH2, IGF1R, JAK2, KDM6A, KDR, KIF5B, KIT, KRAS, LRP1B, MAP2K1, MAP2K4, MCL1, MDM2, MDM4, MET, MGMT, MLL, MPL, MSH6, MTOR, MYC, NF1, NF2, NKX2-1, NOTCH1, NPM, NRAS, PDGFRA, PIK3CA, PIK3R1, PML, PTEN, PTPRD, RARA, RB1, RET, RICTOR, ROS1, RPTOR, RUNX1, SMAD4, SMARCA4, SOX2, STK11, TET2, TP53, TSC1, TSC2, or VHL. In some embodiments, each of the one or more genes with an increased expression is selected from ABL1, AKT1, AKT2, ALK, APC, AR, ARID1A, ASXL1, ATM, AURKA, BAP, BAP1, BCL2L11, BCR, BRAF, BRCA1, BRCA2, CCND1, CCND2, CCND3, CCNE1, CDH1, CDK4, CDK6, CDK8, CDKN1A, CDKN1B, CDKN2A, CDKN2B, CEBPA, CTNNB1, DDR2, DNMT3A, E2F3, EGFR, EML4, EPHB2, ERBB2, ERBB3, ESR1, EWSR1, FBXW7, FGF4, FGFR1, FGFR2, FGFR3, FLT3, FRS2, HIF1A, HRAS, IDH1, IDH2, IGF1R, JAK2, KDM6A, KDR, KIF5B, KIT, KRAS, LRP1B, MAP2K1, MAP2K4, MCL1, MDM2, MDM4, MET, MGMT, MLL, MPL, MSH6, MTOR, MYC, NF1, NF2, NKX2-1, NOTCH1, NPM, NRAS, PDGFRA, PIK3CA, PIK3R1, PML, PTEN, PTPRD, RARA, RB1, RET, RICTOR, ROS1, RPTOR, RUNX1, SMAD4, SMARCA4, SOX2, STK11, TET2, TP53, TSC1, TSC2, or VHL. In some embodiments, the one or more genes with an increased expression is detected in the patient's ctDNA or cancer and the one or more genes is selected from AKT1, AKT2, BRAF, CDK4, CDK6, PIK3CA, PIK3R1, or mTOR.

In one aspect, disclosed herein are methods of monitoring a patient on a breast cancer treatment by (a) determining quantitative measures of the mutant allele frequency (MAF) of circulating tumor DNA (ctDNA) of at least one gain of function missense mutation within the ligand binding domain (LBD) of the Estrogen Receptor 1 (ESR1) gene (ESR1 ctDNA) in a biological sample of the patient, wherein the quantitative measures are performed over a period of time at predetermined intervals; and (b) determining a positive predictive value (PPV) for clinical benefit (CB, defined as stable disease ≥24 weeks, or confirmed partial or complete response) wherein the PPV indicates responsiveness to the cancer treatment, wherein the cancer treatment comprises an effective amount of lasofoxifene or a pharmaceutically acceptable salt thereof and an effective amount of a CDK4/6 inhibitor (CDK4/6i). In some embodiments, the method further comprises determining that the patient has at least one gain of function missense mutation within the ligand binding domain (LBD) of the Estrogen Receptor 1 (ESR1) gene. In certain embodiments, the at least one gain of function missense mutation is selected from D538, Y537, L536, P535, V534, L469, 5463, V392, and E380. In some embodiments, the gain of function mutation is D538G, Y537N, or Y537S. In some embodiments, the patient has decreased MAF post-treatment as determined at 4 weeks. In some embodiments, the patient has decreased MAF post-treatment as determined at 24 weeks.

In some embodiments, the patient is a postmenopausal woman. In some embodiments, the patient is a premenopausal woman. In some embodiments, the patient has osteoporosis or a higher risk of osteoporosis.

In various embodiments, the patient derives clinical benefit (CB, defined as stable disease ≥24 weeks, or confirmed partial or complete response) with stable disease for at least 24 weeks, 28 weeks, 32 weeks, 36 weeks, 40 weeks, 44 weeks, 48 weeks, 52 weeks, 56 weeks, 60 weeks, 64 weeks, 68 weeks, 72 weeks, 74 weeks, 78 weeks, 82 weeks, 86 weeks, 88 weeks, or longer in response to treatment with lasofoxifene in combination with a CDK4/6i (e.g., palbociclib, ribociclib, abemaciclib). In various embodiments, the patient derives an average longer duration of stable disease than on preceding second-line or third-line therapies by at least about 20%, 15%, or 10% in response to treatment with lasofoxifene in combination with a CDK4/6i (e.g., palbociclib, ribociclib, abemaciclib). In some embodiments, the patient has had prior treatment of one or more CDK4/6i (e.g., palbociclib, ribociclib, abemaciclib). In some embodiments, the breast cancer has progressed on one or more prior endocrine therapies selected from a selective ER degrader (SERD), a selective ER modulator (SERM), optionally a SERM other than lasofoxifene, an aromatase inhibitor (AI), a mTOR inhibitor, and/or a PI3K inhibitor. In some embodiments, the breast cancer has progressed on a prior treatment with fulvestrant. In some embodiments, the breast cancer has progressed on a prior treatment with sirolimus, temsirolimus, everolimus, or ridaforolimus. In some embodiments, the breast cancer has progressed on a prior treatment with everolimus. In some embodiments, the breast cancer has progressed on a prior treatment with an mTOR inhibitor and a CDK4/6i (e.g., palbociclib, ribociclib, abemaciclib). In some embodiments, the breast cancer has progressed on a prior treatment with everolimus and palbociclib.

### 5.1.4. Prior Therapy

### 5.1.4.1 Prior Endocrine Therapy

In various embodiments of the methods provided herein, the patient has previously been treated with one or more lines of endocrine therapy. In certain embodiments, the patient's cancer has relapsed or progressed after the previous therapy.

In some embodiments, the prior endocrine therapy is administration of a selective ER modulator (SERM) other than lasofoxifene. In various embodiments, the SERM is selected from tamoxifen, raloxifene, toremifene, ospemifene, broparestrol, bazedoxifene, and ormeloxifene. In certain embodiments, the prior endocrine therapy is administration of tamoxifen.

In some embodiments, the prior endocrine therapy is administration of a selective ER degrader (SERD). In some embodiments, the selective ER degrader is selected from fulvestrant, elacestrant (RAD1901), ARN-810 (GDC-0810), giredestrant (GDC-9545), amcenestrant (SAR439859), rintodestrant (G1T48), LSZ102, imlunestrant (LY3484356), zN-c5, D-0502, SHR9549, camizestrant (AZD9833), and AZD9496. In certain embodiments, the prior endocrine therapy is administration of fulvestrant.

In some embodiments, the prior endocrine therapy is administration of an aromatase inhibitor (AI). In some embodiments, the aromatase inhibitor is selected from exemestane (Aromasin®), letrozole (Femara®), and anastrozole (Arimidex®).

In some embodiments, the prior endocrine therapy is ovarian suppression. In various embodiments, the ovarian suppression is achieved by oophorectomy or administration of a GnRH antagonist.

In some embodiments, the patient's cancer has relapsed or progressed after tamoxifen treatment. In some embodiments, the patient's cancer has relapsed or progressed after fulvestrant treatment. In some embodiments, the patient's cancer has relapsed or progressed after aromatase inhibitor (AI) treatment. In some embodiments, the patient's cancer has relapsed or progressed after AI treatment in combination with a CDK4/6i. In certain embodiments, the patient's cancer has relapsed or progressed after AI treatment in combination with palbociclib or ribociclib. In some of these embodiments, the patient's cancer has relapsed or progressed after multiple lines of endocrine therapy treatment.

### 5.1.4.2 Prior Endocrine Treatment for Metastatic Disease

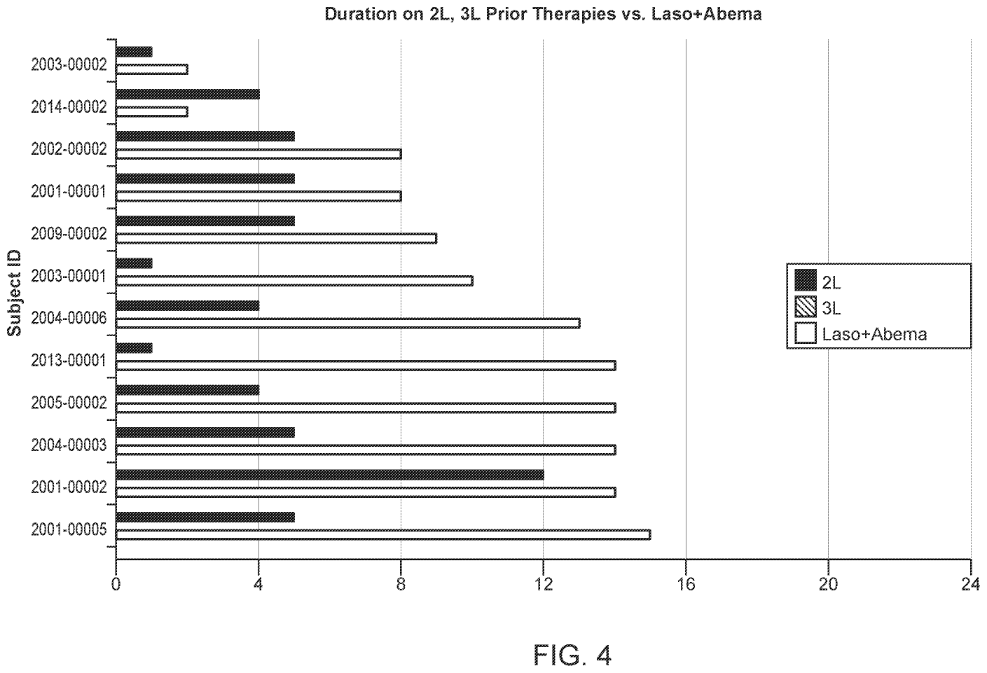

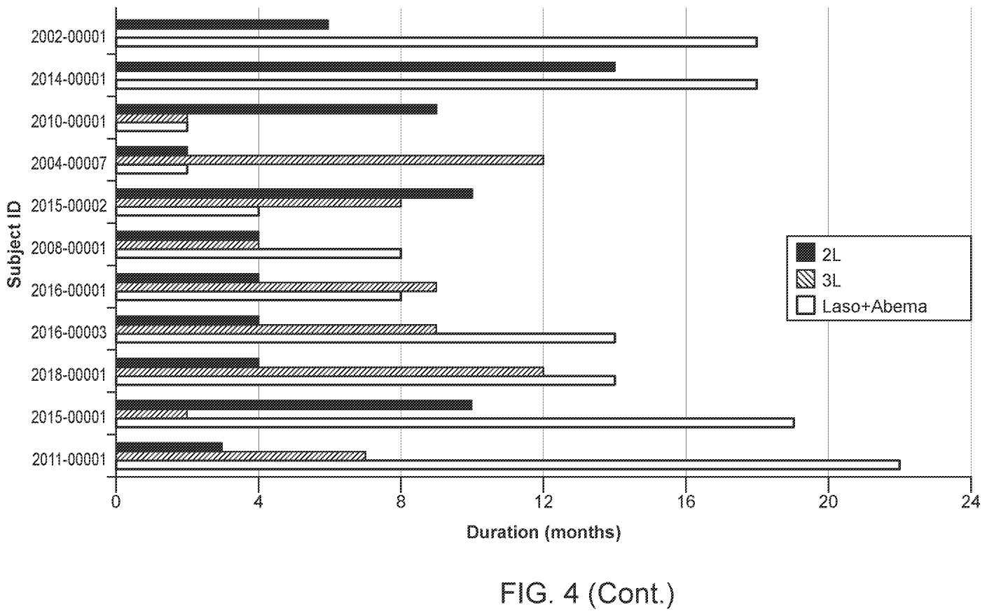

In certain embodiments, the patient's ER⁺ breast cancer is metastatic and has progressed on a first (1L), second line (2L), or third line (3L) of treatment for metastatic disease. In some embodiments, the metastatic disease is local metastasis or metastasis to lymph nodes, or metastasis to visceral organs (e.g., lung, pleural effusion, liver, ascites, CNS). In some embodiments, the patient's cancer has relapsed or progressed after one or more second line or third line (3L) cancer therapies, as illustrated in FIG. 4. In some embodiments, the patient's cancer has relapsed or progressed after treatment of metastatic disease with at least one CDK 4/6 inhibitor and at least one of an endocrine therapy, a mammalian target of rapamycin (mTOR) inhibitor, a phosphatidylinositol-3-kinase (PI3K) inhibitor, a heat shock protein 90 (HSP90) inhibitor, a Poly (ADP-ribose) polymerase (PARP) inhibitor, an AKT inhibitor, or a histone deacetylase (HDAC) inhibitor. In some embodiments, the patient's metastatic breast cancer has relapsed or progressed after treatments of at least one of tamoxifen, fulvestrant, capecitabine, everolimus, alpelisib, talazoparib, palbociclib, ribociclib, or abemaciclib, alone or in combination.

In certain embodiments, progression has occurred on one or two of the following prior treatments for metastatic breast cancer: aromatase inhibitor (AI) and/or fulvestrant either as monotherapy or in combination with any commercially approved CDK 4/6 inhibitor (CDKi); and/or the combination of fulvestrant and alpelisib; and/or tamoxifen; and/or the combination of exemestane/everolimus. In some embodiments, the prior-administered CDK 4/6 inhibitor is abemaciclib, ribociclib, or palbociclib. In some embodiments, progression of metastatic cancer has occurred on at least a prior treatment of abemaciclib, palbociclib, or ribociclib. In some embodiments, progression of metastatic cancer has occurred on a prior abemaciclib treatment. In some embodiments, progression for metastatic cancer has occurred on a prior palbociclib treatment. In some embodiments, progression for metastatic cancer has occurred on a prior ribociclib treatment. In some embodiments, the cancer has metastasized to visceral organs.

In certain embodiments, the metastatic cancer has progressed while on a non-steroid aromatase inhibitor (AI); a SERD (e.g., fulvestrant); AI in combination with a CDK4/6 inhibitor; or a SERD (e.g., fulvestrant) in combination with a CDK4/6 inhibitor.

In certain embodiments, progression has occurred on a CDK 4/6 inhibitor either as monotherapy or combination therapy.

### 5.2. Pharmaceutical Compositions

Methods for treatment of estrogen receptor positive (ER⁺) breast cancer described herein comprise administering to the patient an effective amount of lasofoxifene or a pharmaceutically acceptable salt thereof and an effective amount of a CDK4/6 inhibitor (CDK4/6i).

In some embodiments, lasofoxifene is administered as lasofoxifene tartrate.

In some embodiments the CDK4/6i is selected from palbociclib, abemaciclib, and ribociclib. In certain embodiments, the CDK4/6i is abemaciclib.

The term “pharmaceutically acceptable salt” refers to non-toxic pharmaceutically acceptable salts. Other salts well known to those in the art may, however, be used. Representative organic or inorganic acids include, but are not limited to, hydrochloric, hydrobromic, hydriodic, perchloric, sulfuric, nitric, phosphoric, acetic, propionic, glycolic, lactic, succinic, maleic, fumaric, malic, tartaric, citric, benzoic, mandelic, methanesulfonic, hydroxyethanesulfonic, benzenesulfonic, oxalic, pamoic, 2-naphthalenesulfonic, p-toluenesulfonic, cyclohexanesulfamic, salicylic, saccharinic or trifluoroacetic acid. Representative organic or inorganic bases include, but are not limited to, basic or cationic salts such as benzathine, chloroprocaine, choline, diethanolamine, ethylenediamine, meglumine, procaine, aluminum, calcium, lithium, magnesium, potassium, sodium and zinc.

Embodiments also include prodrugs of the active compounds disclosed herein. In general, such prodrugs will be functional derivatives of the compounds which are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the subject. Conventional procedures for the selection and preparation of suitable prodrug derivatives are described, for example, in “Design of Prodrugs”, H. Bundgaard, Elsevier, 1985.

Some of the crystalline forms for the compounds exist as polymorphs and as such are intended to be included in the present invention. In addition, some of the solid forms of the active compounds exist as solvates e.g., with water (i.e., hydrates) or common organic solvents, and such solvates are encompassed by embodiments of the present invention.

Where the processes for the preparation of the active compounds administered in the presently provided methods give rise to mixtures of stereoisomers, in some embodiments, these isomers are separated by conventional techniques such as preparative chromatography. In some embodiments, the compounds are prepared in racemic form or as individual enantiomers or diastereomers by either stereospecific synthesis or by resolution. In some embodiments, the compounds are resolved into their component enantiomers or diastereomers by standard techniques, such as the formation of stereoisomeric pairs by salt formation with an optically active base, followed by fractional crystallization and regeneration of the free acid. In some embodiments, the compounds are resolved by formation of stereoisomeric esters or amides, followed by chromatographic separation and removal of the chiral auxiliary. Alternatively, the compounds are resolved using a chiral HPLC column. It is to be understood that compositions comprising all stereoisomers, racemic mixtures, diastereomers, cis-trans isomers, and enantiomers thereof are encompassed by embodiments herein.

In certain embodiments, the active compounds are formulated in separate pharmaceutical compositions. In addition to the active compound, the pharmaceutical formulation or composition further comprises one or more of a pharmaceutically acceptable excipient, carrier, buffer, stabilizer or other material well known to those skilled in the art. Such materials should be non-toxic and should not interfere with the efficacy of the active ingredient. The precise nature of the carrier or other material depends on the route of administration, e.g. oral, intravenous, transdermal, vaginal topical, or vaginal ring.

In certain embodiments, pharmaceutical compositions for oral administration are in tablet, capsule, powder or liquid form. In certain embodiments, the tablet includes a solid carrier such as gelatin or an adjuvant. In certain embodiments, the liquid pharmaceutical composition comprises a liquid carrier such as water, petroleum, animal oil, vegetable oil, mineral oil or synthetic oil. Physiological saline solution, dextrose or other saccharide solution or glycols such as ethylene glycol, propylene glycol or polyethylene glycol can also be included.

For parenteral administration, the composition is in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required.

### 5.3. Treatment Regimens

In the methods described herein, the terms “treating”, “treatment”, and grammatical variations thereof are used in the broadest sense understood in the clinical arts. Accordingly, the terms do not require cure or complete remission of disease, and encompass obtaining any clinically desired pharmacologic and/or physiologic effect. In certain embodiments, the effect is partial or complete response of the breast cancer, slowing or inhibiting the progression of the cancer; or causing regression of the cancer.

The term “effective amount” as used herein with respect to the combination therapy means the individual dosages of each of lasofoxifene and CDK4/6i that, in combination, produce the desired effect for which they are administered.

### 5.3.1. Combination administration

In some embodiments of the provided methods, lasofoxifene or pharmaceutically acceptable salt thereof and the CDK4/6i (e.g., abemaciclib, palbociclib, ribociclib) are administered as separate dosage forms. In certain of these embodiments, lasofoxifene or salt thereof and the CDK4/6i are separately administered at the same time (simultaneously). In some other embodiments, lasofoxifene or salt thereof and the CDK4/6i are administered as separate dosage forms at separate times (e.g., sequentially, or on unrelated schedules).

In certain embodiments, lasofoxifene is administered in a single dosage form comprising both lasofoxifene or salt thereof and a CDK4/6i. In certain embodiments, the CDK4/6i is abemaciclib.

### 5.3.1.1 Administration of Lasofoxifene

In various embodiments of the methods of treatment described above, lasofoxifene or pharmaceutically acceptable salt thereof is administered by oral administration.

In some embodiments, lasofoxifene or pharmaceutically acceptable salt thereof is administered to the patient by oral administration (per os; p.o.) at a lasofoxifene dosage of about 0.5 mg/day per os to about 10 mg/day per os, such as about 0.5 mg/day per os to about 5 mg/day per os, about 1 mg/day per os to about 5 mg/day per os, about 2 mg/day per os to about 5 mg/day per os, about 3 mg/day per os to about 5 mg/day per os, about 4 mg/day per os to about 5 mg/day per os, about 0.5 mg/day per os to about 4 mg/day per os, about 1 mg/day per os to about 4 mg/day per os, about 2 mg/day per os to about 4 mg/day per os, about 3 mg/day per os to about 4 mg/day per os, about 0.5 mg/day per os to about 3 mg/day per os, about 1 mg/day per os to about 3 mg/day per os, about 2 mg/day per os to about 3 mg/day per os, about 0.5 mg/day per os to about 2 mg/day per os, about 1 mg/day per os to about 2 mg/day per os, or about 0.5 mg/day per os to about 1 mg/day per os.

In some embodiments, lasofoxifene or pharmaceutically acceptable salt thereof is administered at about 0.5 mg lasofoxifene/day per os. In some embodiments, lasofoxifene or pharmaceutically acceptable salt thereof is administered at about 1 mg lasofoxifene/day per os. In some embodiments, lasofoxifene or pharmaceutically acceptable salt thereof at about 1.5 mg lasofoxifene/day per os, about 2 mg lasofoxifene/day per os, 2.5 mg lasofoxifene/day per os, about 3 mg lasofoxifene/day er os, about 3.5 mg lasofoxifene/day per os, about 4 mg lasofoxifene/day per os, about 4.5 mg lasofoxifene/day per os, about 5 mg lasofoxifene/day per os, about 6 mg lasofoxifene/day per os, about 7 mg lasofoxifene/day per os, about 8 mg lasofoxifene/day per os, about 9 mg lasofoxifene/day per os, or about 10 mg lasofoxifene/day per os. In some other embodiments, lasofoxifene or pharmaceutically acceptable salt thereof is administered at a dose of more than 10 mg lasofoxifene/day per os.

In some embodiments, lasofoxifene or pharmaceutically acceptable salt thereof is administered at 0.5 mg/day lasofoxifene to 10 mg/day. In some embodiments, lasofoxifene or pharmaceutically acceptable salt thereof is administered at 0.5 mg lasofoxifene/day, 1 mg lasofoxifene/day, 1.5 mg lasofoxifene/day, 2 mg lasofoxifene/day, 2.5 mg lasofoxifene/day, 3 mg lasofoxifene/day, 3.5 mg lasofoxifene/day, 4 mg lasofoxifene/day, 5 mg lasofoxifene/day, mg lasofoxifene/day, 6 mg lasofoxifene/day, 6.5 mg lasofoxifene/day, 7 mg lasofoxifene/day, 7.5 mg lasofoxifene/day, 8 mg lasofoxifene/day, 8.5 mg lasofoxifene/day, 9 mg lasofoxifene/day, 9.5 mg lasofoxifene/day, or 10 mg lasofoxifene/day. In currently preferred embodiments, lasofoxifene or pharmaceutically acceptable salt thereof is administered orally at 5 mg lasofoxifene/day. In particular embodiments, lasofoxifene tartrate is administered orally at 5 mg lasofoxifene/day.

In certain embodiments, lasofoxifene is administered once every day. In certain embodiments, lasofoxifene is administered once every two days. In certain embodiments, lasofoxifene is administered once every three days. In certain embodiments, lasofoxifene is administered once every four days. In certain embodiments, lasofoxifene is administered once every five days. In certain embodiments, lasofoxifene is administered once every six days. In certain embodiments, lasofoxifene is administered once every week. In certain embodiments, lasofoxifene is administered once every two weeks. In certain embodiments, lasofoxifene is administered once every three weeks. In certain embodiments, lasofoxifene is administered once every month.

In some embodiments, lasofoxifene is administered to the patient by vaginal ring administration. In some of these embodiments, lasofoxifene is administered once every two weeks. In some of these embodiments, lasofoxifene is administered once every three weeks. In some of these embodiments, lasofoxifene is administered once every month. In some of these embodiments, lasofoxifene is administered once every two months. In some of these embodiments, lasofoxifene is administered once every three months. In some of these embodiments, lasofoxifene is administered once every four months.

In some embodiments, lasofoxifene is administered to the ER⁺ breast cancer patient until the patient's cancer progresses on therapy, is in full remission, or until the side effects are intolerable.

### 5.3.1.2 Administration of Abemaciclib

In each of the methods of treatment described above, the patient is administered lasofoxifene (e.g., lasofoxifene tartrate) in combination with a CDK4/6 inhibitor. In typical embodiments, the CDK4/6 inhibitor is selected from palbociclib, ribociclib, and abemaciclib. In currently preferred embodiments, the patient is administered lasofoxifene tartrate orally at 5 mg lasofoxifene/day and abemaciclib at 50 mg to 200 mg BID, 100 mg to 200 mg BID, or 150 mg BID.

In various embodiments, abemaciclib is administered orally.

In some embodiments, abemaciclib is administered at about 25 mg/day per os to about 600 mg/day per os, such as about 50 mg/day per os to about 200 mg/day per os, e.g., about 25 mg/day per os, about 50 mg/day per os, about 100 mg/day per os, about 150 mg/day per os, about 200 mg/day per os, about 250 mg/day per os, about 300 mg/day per os, about 350 mg/day per os, about 400 mg/day per os, about 450 mg/day per os, or about 600 mg/day per os.

In some embodiments, abemaciclib is administered once every day. In certain embodiments, abemaciclib is administered twice a day.

In certain embodiments, abemaciclib is administered twice daily (BID). In typical embodiments, the daily dose is administered in two equally divided doses. In some embodiments, abemaciclib 300 mg/day per os is administered as two separate 150mg doses orally (e.g., one 150 mg tablet in the morning and one 150 mg tablet in the evening).

In some embodiments, abemaciclib is administered in a starting dose that is subsequently adjusted downward according to a first dose reduction schedule. In certain embodiments, the abemaciclib dose is subsequently reduced according to a second dose reduction schedule. In certain embodiments, the abemaciclib dose is subsequently reduced according to a third dose reduction schedule. In certain of these embodiments, each dose reduction is by 50 mg per dose. For example, a 300 mg/day dose (150 mg per os administered twice daily) is reduced according to a first dose reduction such that 200 mg/day dose is administered twice daily (100 mg per os administered twice daily).

### 5.4. Clinical Endpoints

In various embodiments, the methods comprise administering the combination of lasofoxifene and a CDK4/6i, wherein the combination is effective to treat ER⁺ breast cancer having at least one ESR1 mutation. In certain embodiments, the method is effective to reduce progression of ER⁺ breast cancer having at least one ESR1 mutation.

In certain embodiments, the combination is effective to increase the disease-free survival of the ER⁺ breast cancer patient. In certain embodiments, the combination is effective to reduce recurrence of ER⁺ breast cancer. In certain embodiments, the combination is effective to increase time until recurrence of ER⁺ breast cancer. In certain embodiments, the combination is effective to reduce metastasis of ER⁺ breast cancer. In certain embodiments, the combination is effective to increase duration of progression-free survival of the ER⁺ breast cancer patient. In certain embodiments, the comparison to determine efficacy is made against the standard of care.

In some embodiments, the method provided herein increases disease-free survival, reduces recurrence, increases time to recurrence, reduces metastasis, and/or increases duration of progression-free survival in patients with ER⁺ locally advanced or metastatic breast cancer that has one or more of the ESR1 mutations discussed herein. In some embodiments, the method reduces the selective pressure and prevents the expansion of the endocrine resistant clones in ER⁺ locally advanced or metastatic breast cancer during treatment.

In some embodiments, lasofoxifene or salt thereof and abemaciclib are administered to the patient until the patient's cancer is in full remission, progresses on therapy, or until the side effects are intolerable.

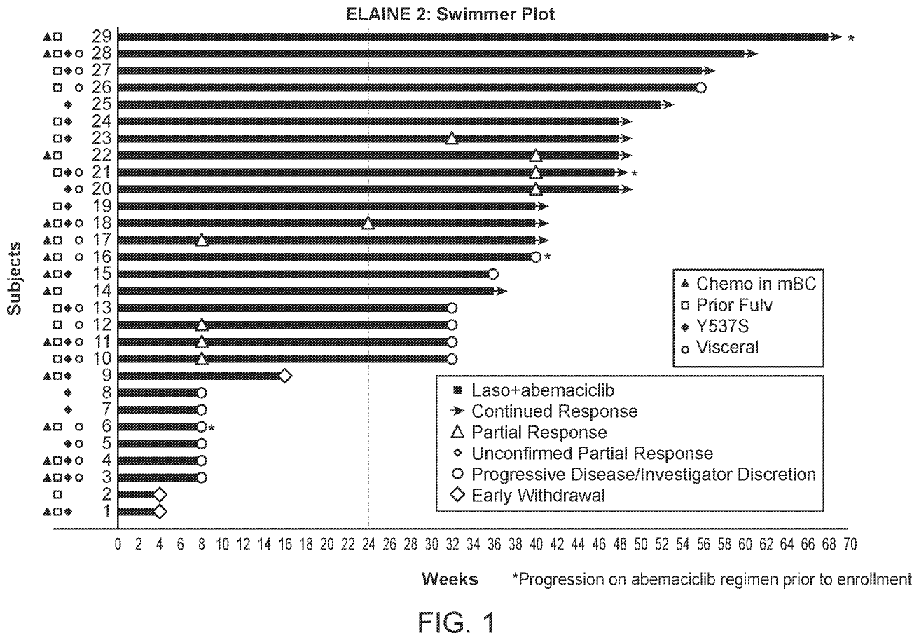

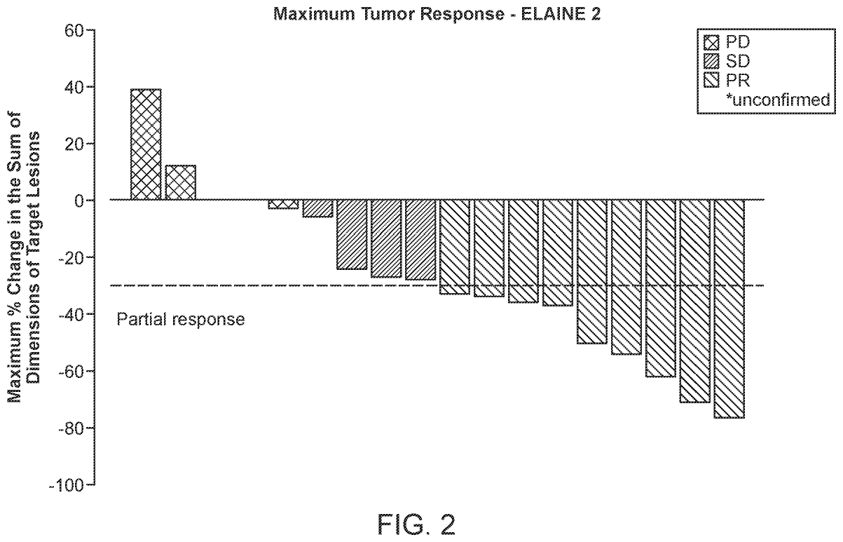

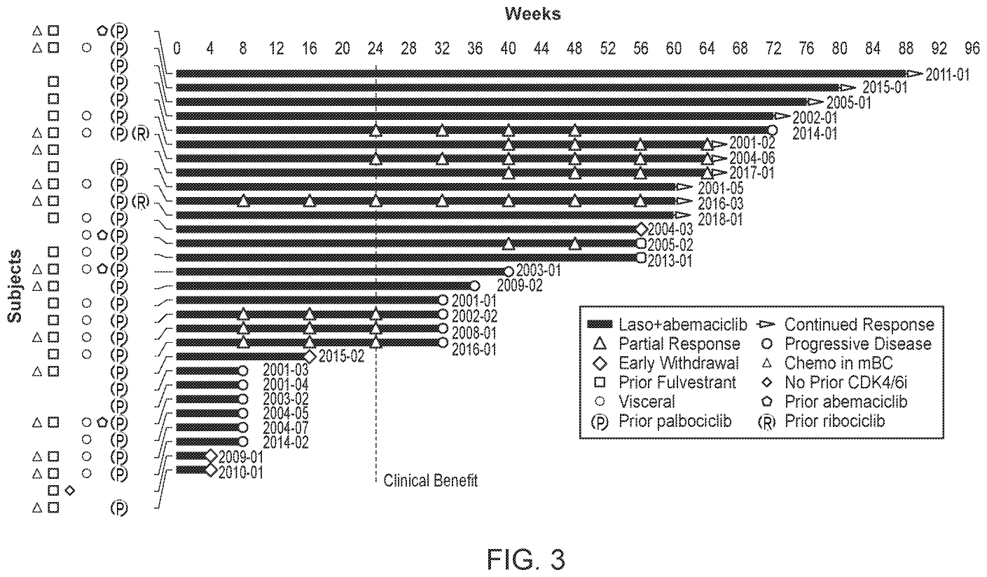

In some embodiments, lasofoxifene or pharmaceutically acceptable salt thereof and a CDK 4/6 inhibitor (e.g., abemaciclib, ribociclib, palbociclib) are administered at doses and for a time effective to provide clinical benefit (CB, defined as stable disease ≥24 weeks, or confirmed partial or complete response) in patients whose cancer has relapsed or progressed after one or more 2L or 3L cancer therapies, as illustrated in FIGS. 1 and 3 and in Examples 2 and 3. In some embodiments, lasofoxifene and a CDK 4/6 inhibitor (e.g., abemaciclib, ribociclib, palbociclib) are administered at doses and for a time effective to provide greater duration of response than any preceding 2L or 3L therapies. In some embodiments, lasofoxifene and a CDK 4/6 inhibitor (e.g., lasofoxifene and abemaciclib) confers CB with stable disease for at least 24 weeks, at least 28 weeks, at least 32 weeks, at least 36 weeks, at least 40 weeks, at least 44 weeks, at least 48 weeks, at least 52 weeks, at least 56 weeks, at least 60 weeks, at least 64 weeks, at least 68 weeks, at least 72 weeks, at least 76 weeks, at least 80 weeks, at least 84 weeks, at least 88 weeks, or longer. In some embodiments, lasofoxifene plus abemaciclib treatment confers stable disease for at least 1 month, at least 2 months, at least 3 months, at least 4 months, at least 5 months, at least 6 months, at least 7 months, at least 8 months, at least 9 months, at least 10 months, at least 11 months, at least 12 months, or longer. In some embodiments, lasofoxifene and a CDK 4/6 inhibitor (e.g., lasofoxifene/abemaciclib) treatment confers CB (defined as stable disease ≥24 weeks, or confirmed partial or complete response) for at least 1 year, at least 2 years, at least 3 years, at least 4 years, at least 5 years, at least 6 years, at least 7 years, at least 8 years, at least 9 years, at least 10 years, at least 11 years, at least 12 years, or longer. In some embodiments, lasofoxifene and CDK 4/6 inhibitor (e.g., lasofoxifene/abemaciclib) treatment confers CB with complete or partial response where the patient's tumor is significantly reduced as illustrated in FIG. 2.

### 5.5. Specific Embodiments

Embodiment 1. A method of reducing the progression of breast cancer in a patient, comprising:
administering to the patient an effective amount of lasofoxifene or a pharmaceutically acceptable salt thereof and an effective amount of a CDK4/6 inhibitor (CDK4/6i), wherein the breast cancer:
(i) is estrogen receptor positive (ER⁺);
(ii) has at least one gain of function missense mutation within the ligand binding domain (LBD) of the Estrogen Receptor 1 (ESR1) gene; and
(iii) has progressed during prior CDK4/6 inhibitor therapy.

Embodiment 2. The method of embodiment 1, wherein the ER⁺ breast cancer is HER2⁻.

Embodiment 3. The method of embodiment 1 or embodiment 2, wherein the ER⁺ breast cancer is locally advanced.

Embodiment 4. The method of embodiment 1 or embodiment 2, wherein the ER⁺ breast cancer is metastatic.

Embodiment 5. The method of any one of embodiments 1-4, wherein lasofoxifene is administered as lasofoxifene tartrate.

Embodiment 6. The method of any one of embodiments 1-5, wherein lasofoxifene is administered orally at 5 mg/day.

Embodiment 7. The method of any one of embodiments 1-6, wherein the CDK4/6i administered to the patient is selected from palbociclib, ribociclib, and abemaciclib.

Embodiment 8. The method of embodiment 7, wherein the CDK4/6i administered to the patient is abemaciclib.

Embodiment 9. The method of embodiment 8, wherein abemaciclib is administered orally at 50 mg to 200 mg BID.

Embodiment 10. The method of embodiment 9, wherein abemaciclib is administered orally at 100 mg to 200 mg BID.

Embodiment 11. The method of embodiment 10, wherein abemaciclib is administered orally at 150 mg BID.

Embodiment 12. The method of embodiment 1, wherein the prior administered CDK4/6 inhibitor is selected from palbociclib, ribociclib, and abemaciclib.

Embodiment 13. The method of embodiment 12, wherein the prior administered CDK4/6 inhibitor is abemaciclib.

Embodiment 14. The method of any one of embodiments 1-13, wherein the cancer has previously been determined to have at least one gain of function missense mutation within the ligand binding domain (LBD) of the Estrogen Receptor 1 (ESR1) gene.

Embodiment 15. The method of any one of embodiments 1-14, further comprising the earlier step of:
determining that the patient has at least one gain of function missense mutation within the ligand binding domain (LBD) of the Estrogen Receptor 1 (ESR1) gene.

Embodiment 16. The method of any one of embodiments 1-15, wherein the at least one of gain of function missense mutation is in any one of amino acids D538, Y537, L469, L536, P535, V534, S463, V392, and E380.

Embodiment 17. The method of embodiment 16, wherein the at least one gain of function missense mutation is in amino acid D538.

Embodiment 18. The method of embodiment 17, wherein the mutation is D538G.

Embodiment 19. The method of embodiment 16, wherein the at least one gain of function missense mutation is in the amino acid Y537.

Embodiment 20. The method of embodiment 19, wherein the mutation is Y537S, Y537N, Y537C, or Y537Q.

Embodiment 21. The method of embodiment 20, wherein the mutation is Y537C.

Embodiment 22. The method of embodiment 16, wherein the at least one gain of function missense mutation is in the amino acid L469.

Embodiment 23. The method of embodiment 22, wherein the mutation is L469V.

Embodiment 24. The method of embodiment 16, wherein the at least one gain of function missense mutation is in the amino acid L536.

Embodiment 25. The method of embodiment 24, wherein the mutation is L536R or L536Q.

Embodiment 26. The method of embodiment 16, wherein the at least one gain of function missense mutation is in the amino acid P535.

Embodiment 27. The method of embodiment 26, wherein the mutation is P535H.

Embodiment 28. The method of embodiment 16, wherein the at least one gain of function missense mutation is in the amino acid V534.

Embodiment 29. The method of embodiment 28, wherein the mutation is V534E.

Embodiment 30. The method of embodiment 16, wherein the at least one gain of function missense mutation is in the amino acid 5463.

Embodiment 31. The method of embodiment 30, wherein the mutation is S463P.

Embodiment 32. The method of embodiment 16, wherein the at least one gain of function missense mutation is in the amino acid V392.

Embodiment 33. The method of embodiment 32, wherein the mutation is V392I.

Embodiment 34. The method of embodiment 16, wherein the at least one gain of function missense mutation is in the amino acid E380.

Embodiment 35. The method of embodiment 34, wherein the mutation is E380Q.

Embodiment 36. The method of embodiment 4, wherein the ER⁺ breast cancer is visceral metastatic.

Embodiment 37. The method of any one of embodiments 1-36, wherein the breast cancer had progressed on one or more prior endocrine therapies.

Embodiment 38. The method of embodiment 37, wherein the prior endocrine therapy is a selective ER degrader (SERD), a selective ER modulator (SERM), optionally a SERM other than lasofoxifene, an aromatase inhibitor (AI), a mTOR inhibitor, and/or a PI3K inhibitor.

Embodiment 39. The method of embodiment 38, wherein the SERD is fulvestrant.

Embodiment 40. The method of any one of embodiments 1-39, wherein the patient derives clinical benefit with stable disease for at least 24 weeks, 28 weeks, 32 weeks, 36 weeks, 40 weeks, 44 weeks, 48 weeks, 52 weeks, 56 weeks, 60 weeks, 64 weeks, 68 weeks, 72 weeks, 74 weeks, 78 weeks, 82 weeks, 86 weeks, or longer following commencement of treatment with lasofoxifene or a pharmaceutically acceptable salt thereof and a CDK4/6i.

Embodiment 41. The method of embodiment 40, wherein the patient has complete or partial response to the treatment with lasofoxifene or a pharmaceutically acceptable salt thereof and a CDK4/6i.

Embodiment 42. The method of any one of embodiments 1-41, wherein the patient derives a longer duration of stable disease than on the patient's preceding second-line or third-line therapies by at least about 20%, 15%, or 10%.

Embodiment 43. A method of reducing the progression of breast cancer in a patient, comprising:
administering to the patient an effective amount of lasofoxifene or a pharmaceutically acceptable salt thereof and an effective amount of a CDK4/6 inhibitor (CDK4/6i),
wherein the breast cancer:
(i) is estrogen receptor positive (ER⁺);
(ii) has at least one gain of function missense mutation within the ligand binding domain (LBD) of the Estrogen Receptor 1 (ESR1) gene; and
(iii) has an oncogenic mutation in one or more genes other than the ESR1 gene.

Embodiment 44. The method of embodiment 43, wherein the breast cancer has progressed during prior CDK4/6 inhibitor therapy.

Embodiment 45. The method of embodiment 43 or 44, wherein the ER⁺breast cancer is HER2⁻.

Embodiment 46. The method of embodiment 45, wherein the ER⁺ breast cancer is locally advanced.

Embodiment 47. The method of embodiment 45, wherein the ER⁺ breast cancer is metastatic.

Embodiment 48. The method of any one of embodiments 43-47, wherein lasofoxifene is administered as lasofoxifene tartrate.

Embodiment 49. The method of any one of embodiments 43-48, wherein lasofoxifene is administered orally at 5 mg/day.

Embodiment 50. The method of any one of embodiments 43-49, wherein the CDK4/6i administered to the patient is selected from palbociclib, ribociclib, and abemaciclib.

Embodiment 51. The method of embodiment 50, wherein the CDK4/6i administered to the patient is abemaciclib.

Embodiment 52. The method of embodiment 51, wherein abemaciclib is administered orally at 50 mg to 200 mg BID.

Embodiment 53. The method of embodiment 52, wherein abemaciclib is administered orally at 100 mg to 200 mg BID.

Embodiment 54. The method of embodiment 53, wherein abemaciclib is administered orally at 150 mg BID.

Embodiment 55. The method of embodiment 44, or of any one of embodiments 45-54 as dependent from embodiment 44, wherein the prior administered CDK4/6 inhibitor is selected from palbociclib, ribociclib, and abemaciclib.

Embodiment 56. The method of embodiment 55, wherein the prior administered CDK4/6 inhibitor is abemaciclib.

Embodiment 57. The method of any one of embodiments 43-56, further comprising the preceding step of detecting oncogenic mutations in the one or more genes with an oncogenic mutation in circulating tumor DNA (ctDNA).

Embodiment 58. The method of embodiment 57, wherein at least one of the one or more genes with an oncogenic mutation is selected from HNF1A, TERT, GATA3, CDK12, MAPK3, GNAS, RAF1, RHEB, NTRK3, TP53, PIK3CA, APC, ATM, MET, CCND1, EGFR, ROS1, BRCA2, STK11, FGFR1, ARID1A, CCNE1, AR, ALK, ERBB2, KIT, CDK4, SMAD4, NOTCH1, RB1, BRAF, PTEN, AKT1, CDH1, BRCA1, MYC, CDKN2A, IDH2, MTOR, or PDGFRA.

Embodiment 59. The method of embodiment 58, wherein each of the one or more genes with an oncogenic mutation is selected from HNF1A, TERT, GATA3, CDK12, MAPK3, GNAS, RAF1, RHEB, NTRK3, TP53, PIK3CA, APC, ATM, MET, CCND1, EGFR, ROS1, BRCA2, STK11, FGFR1, ARID1A, CCNE1, AR, ALK, ERBB2, KIT, CDK4, SMAD4, NOTCH1, RB1, BRAF, PTEN, AKT1, CDH1, BRCA1, MYC, CDKN2A, IDH2, MTOR, or PDGFRA.

Embodiment 60. The method of any one of embodiments 43-59, wherein the one or more genes with an oncogenic mutation is selected from HNF1A, TERT, GATA3, CDK12, MAPK3, TP53, PIK3CA, APC, ATM, MET, CCND1, EGFR, ROS1, STK11, FGFR1, ARID1A, CCNE1, AR, ALK, ERBB2, KIT, CDK4, SMAD4, NOTCH1, RB1, BRAF, RAF1, PTEN, AKT1, CDH1, BRCA1, MYC, CDKN2A, or PDGFRA.

Embodiment 61. The method of any one of embodiments 43-59, wherein the one or more genes with an oncogenic mutation is selected from CCND1, FGFR1, CCNE1, AR, ALK, MAPK3, KIT, SMAD4, NOTCH1, RB1, BRAF, RAF1, PTEN, AKT1, CDH1, BRCA1, MYC, CDKN2A, or PDGFRA.

Embodiment 62. The method of any one of embodiments 43-59, wherein the one or more genes with an oncogenic mutation is selected from HNF1A, TERT, GATA3, CDK12, MAPK3, TP53, PIK3CA, APC, ATM, MET, CCND1, EGFR, ROS1, BRCA2, STK11, FGFR1, ARID1A, CCNE1, AR, ALK, ERBB2, KIT, BRAF, or CDK4.

Embodiment 63. The method of embodiment 62, wherein the one or more genes with an oncogenic mutation is selected from TP53, PIK3CA, CCND1, ARID1A, FGFR1, CCNE1 and ERBB2.

Embodiment 64. The method of embodiment 63, wherein the oncogenic mutation is PIK3 CA.

Embodiment 65. The method of any one of embodiments 43-59, wherein the one or more genes with an oncogenic mutation is selected from HNF1A, TERT, GATA3, CDK12, MAPK3, GNAS, RAF1, RHEB, or NTRK3.

Embodiment 66. The method of any one of embodiments 43-65, wherein the cancer has previously been determined to have at least one gain of function missense mutation within the ligand binding domain (LBD) of the Estrogen Receptor 1 (ESR1) gene.

Embodiment 67. The method of any one of embodiments 43-66, further comprising the earlier step of:
determining that the patient has at least one gain of function missense mutation within the ligand binding domain (LBD) of the Estrogen Receptor 1 (ESR1) gene.

Embodiment 68. The method of any one of embodiments 43-67, wherein the at least one of gain of function missense mutation is in any one of amino acids D538, Y537, L469, L536, P535, V534, S463, V392, and E380.

Embodiment 69. The method of embodiment 68, wherein the at least one gain of function missense mutation is in amino acid D538.

Embodiment 70. The method of embodiment 69, wherein the mutation is D538G.

Embodiment 71. The method of embodiment 68, wherein the at least one gain of function missense mutation is in the amino acid Y537.

Embodiment 72. The method of embodiment 71, wherein the mutation is Y537S, Y537N, Y537C, or Y537Q.

Embodiment 73. The method of embodiment 72, wherein the mutation is Y537C. Embodiment 74. The method of embodiment 72, wherein the mutation is Y537S.

Embodiment 75. The method of embodiment 68, wherein the at least one gain of function missense mutation is in the amino acid L469.

Embodiment 76. The method of embodiment 75, wherein the mutation is L469V.

Embodiment 77. The method of embodiment 68, wherein the at least one gain of function missense mutation is in the amino acid L536.

Embodiment 78. The method of embodiment 77, wherein the mutation is L536R or L536Q.

Embodiment 79. The method of embodiment 68, wherein the at least one gain of function missense mutation is in the amino acid P535.

Embodiment 80. The method of embodiment 79, wherein the mutation is P535H.

Embodiment 81. The method of embodiment 68, wherein the at least one gain of function missense mutation is in the amino acid V534.

Embodiment 82. The method of embodiment 81, wherein the mutation is V534E.

Embodiment 83. The method of embodiment 68, wherein the at least one gain of function missense mutation is in the amino acid 5463.

Embodiment 84. The method of embodiment 83, wherein the mutation is S463P.

Embodiment 85. The method of embodiment 68, wherein the at least one gain of function missense mutation is in the amino acid V392.

Embodiment 86. The method of embodiment 85, wherein the mutation is V392I.

Embodiment 87. The method of embodiment 68, wherein the at least one gain of function missense mutation is in the amino acid E380.

Embodiment 88. The method of embodiment 87, wherein the mutation is E380Q.

Embodiment 89. The method of embodiment 47, wherein the ER⁺ breast cancer is visceral metastatic.

Embodiment 90. The method of any one of embodiments 43-89, wherein the breast cancer had progressed on one or more prior endocrine therapies.

Embodiment 91. The method of embodiment 90, wherein the prior endocrine therapy is a selective ER degrader (SERD), a selective ER modulator (SERM), optionally a SERM other than lasofoxifene, an aromatase inhibitor (AI), a mTOR inhibitor, and/or a PI3K inhibitor.

Embodiment 92. The method of embodiment 91, wherein the SERD is fulvestrant.

Embodiment 93. The method of any one of embodiments 43-92, wherein the patient derives clinical benefit with stable disease for at least 24 weeks, 28 weeks, 32 weeks, 36 weeks, 40 weeks, 44 weeks, 48 weeks, 52 weeks, 56 weeks, 60 weeks, 64 weeks, 68 weeks, 72 weeks, 74 weeks, 78 weeks, 82 weeks, 86 weeks, or longer following commencement of treatment with lasofoxifene or a pharmaceutically acceptable salt thereof and a CDK4/6i.

Embodiment 94. The method of embodiment 93, wherein the patient has complete or partial response to the treatment with lasofoxifene or a pharmaceutically acceptable salt thereof and a CDK4/6i.

Embodiment 95. The method of any one of embodiments 43-94, wherein the patient derives a longer duration of stable disease on with lasofoxifene or a pharmaceutically acceptable salt thereof and a CDK4/6i than on the patient's preceding second-line or third-line therapies by at least about 20%, 15%, or 10%.

Embodiment 96. A method of reducing the progression of breast cancer in a patient, comprising:
administering to the patient an effective amount of lasofoxifene or a pharmaceutically acceptable salt thereof and an effective amount of a CDK4/6 inhibitor (CDK4/6i),
wherein the breast cancer:
(i) is estrogen receptor positive (ER⁺);
(ii) has at least one gain of function missense mutation within the ligand binding domain (LBD) of the Estrogen Receptor 1 (ESR1) gene; and
(iii) has increased expression of one or more genes other than the ESR1 gene.

Embodiment 97. The method of embodiment 96, wherein the breast cancer has progressed during prior CDK4/6 inhibitor therapy.

Embodiment 98. The method of embodiment 96 or 97, wherein the ER⁺ breast cancer is HER2⁻.

Embodiment 99. The method of embodiment 98, wherein the ER⁺ breast cancer is locally advanced.

Embodiment 100. The method of embodiment 98, wherein the ER⁺ breast cancer is metastatic.

Embodiment 101. The method of any one of embodiments 96-100, wherein lasofoxifene is administered as lasofoxifene tartrate.

Embodiment 102. The method of any one of embodiments 96-101, wherein lasofoxifene or lasofoxifene tartrate is administered orally at 5 mg lasofoxifene/day.

Embodiment 103. The method of any one of embodiments 96-102, wherein the CDK4/6i administered to the patient is selected from palbociclib, ribociclib, and abemaciclib.

Embodiment 104. The method of embodiment 103, wherein the CDK4/6i administered to the patient is abemaciclib.

Embodiment 105. The method of embodiment 104, wherein abemaciclib is administered orally at 50 mg to 200 mg BID.

Embodiment 106. The method of embodiment 105, wherein abemaciclib is administered orally at 100 mg to 200 mg BID.

Embodiment 107. The method of embodiment 106, wherein abemaciclib is administered orally at 150 mg BID.

Embodiment 108. The method of any one of embodiments 96-107, wherein the prior administered CDK4/6 inhibitor is selected from palbociclib, ribociclib, and abemaciclib.

Embodiment 109. The method of embodiment 108, wherein the prior administered CDK4/6 inhibitor is abemaciclib.

Embodiment 110. The method of any one of embodiments 96-109, further comprising the earlier step of detecting increased expression of the one or more genes other than ESR1 in circulating tumor DNA (ctDNA).

Embodiment 111. The method of embodiment 110, wherein at least one of the one or more genes with increased expression is selected from ABL1, AKT1, AKT2, ALK, APC, AR, ARID1A, ASXL1, ATM, AURKA, BAP, BAP1, BCL2L11, BCR, BRAF, BRCA1, BRCA2, CCND1, CCND2, CCND3, CCNE1, CDH1, CDK4, CDK6, CDK8, CDKN1A, CDKN1B, CDKN2A, CDKN2B, CEBPA, CTNNB1, DDR2, DNMT3A, E2F3, EGFR, EML4, EPHB2, ERBB2, ERBB3, ESR1, EWSR1, FBXW7, FGF4, FGFR1, FGFR2, FGFR3, FLT3, FRS2, HIF1A, HRAS, IDH1, IDH2, IGF1R, JAK2, KDM6A, KDR, KIF5B, KIT, KRAS, LRP1B, MAP2K1, MAP2K4, MCL1, MDM2, MDM4, MET, MGMT, MLL, MPL, MSH6, MTOR, MYC, NF1, NF2, NKX2-1, NOTCH1, NPM, NRAS, PDGFRA, PIK3CA, PIK3R1, PML, PTEN, PTPRD, RARA, RB1, RET, RICTOR, ROS1, RPTOR, RUNX1, SMAD4, SMARCA4, SOX2, STK11, TET2, TP53, TSC1, TSC2, or VHL.

Embodiment 112. The method of embodiment 111, wherein each of the one or more genes with increased expression is selected from ABL1, AKT1, AKT2, ALK, APC, AR, ARID1A, ASXL1, ATM, AURKA, BAP, BAP1, BCL2L11, BCR, BRAF, BRCA1, BRCA2, CCND1, CCND2, CCND3, CCNE1, CDH1, CDK4, CDK6, CDK8, CDKN1A, CDKN1B, CDKN2A, CDKN2B, CEBPA, CTNNB1, DDR2, DNMT3A, E2F3, EGFR, EML4, EPHB2, ERBB2, ERBB3, ESR1, EWSR1, FBXW7, FGF4, FGFR1, FGFR2, FGFR3, FLT3, FRS2, HIF1A, HRAS, IDH1, IDH2, IGF1R, JAK2, KDM6A, KDR, KIF5B, KIT, KRAS, LRP1B, MAP2K1, MAP2K4, MCL1, MDM2, MDM4, MET, MGMT, MLL, MPL, MSH6, MTOR, MYC, NF1, NF2, NKX2-1, NOTCH1, NPM, NRAS, PDGFRA, PIK3CA, PIK3R1, PML, PTEN, PTPRD, RARA, RB1, RET, RICTOR, ROS1, RPTOR, RUNX1, SMAD4, SMARCA4, SOX2, STK11, TET2, TP53, TSC1, TSC2, or VHL.

Embodiment 113. The method of any one of embodiments 96-112, wherein the one or more genes with increased expression is selected from AKT1, AKT2, BRAF, CDK4, CDK6, PIK3CA, PIK3R1, or mTOR.

Embodiment 114. The method of any one of embodiments 96-113, wherein the cancer has previously been determined to have at least one gain of function missense mutation within the ligand binding domain (LBD) of the Estrogen Receptor 1 (ESR1) gene.

Embodiment 115. The method of any one of embodiments 96-114, further comprising the earlier step of:
determining that the patient has at least one gain of function missense mutation within the ligand binding domain (LBD) of the Estrogen Receptor 1 (ESR1) gene.

Embodiment 116. The method of any one of embodiments 96-115, wherein the at least one of gain of function missense mutation is in any one of amino acids D538, Y537, L469, L536, P535, V534, S463, V392, and E380.

Embodiment 117. The method of embodiment 116, wherein the at least one gain of function missense mutation is in amino acid D538.

Embodiment 118. The method of embodiment 117, wherein the mutation is D538G.

Embodiment 119. The method of embodiment 116, wherein the at least one gain of function missense mutation is in the amino acid Y537.

Embodiment 120. The method of embodiment 119, wherein the mutation is Y537S, Y537N, Y537C, or Y537Q.

Embodiment 121. The method of embodiment 120, wherein the mutation is Y537C.

Embodiment 122. The method of embodiment 120, wherein the mutation is Y537S.

Embodiment 123. The method of embodiment 116, wherein the at least one gain of function missense mutation is in the amino acid L469.

Embodiment 124. The method of embodiment 123, wherein the mutation is L469V.

Embodiment 125. The method of embodiment 116, wherein the at least one gain of function missense mutation is in the amino acid L536.

Embodiment 126. The method of embodiment 125, wherein the mutation is L536R or L536Q.

Embodiment 127. The method of embodiment 116, wherein the at least one gain of function missense mutation is in the amino acid P535.

Embodiment 128. The method of embodiment 127, wherein the mutation is P535H.

Embodiment 129. The method of embodiment 116, wherein the at least one gain of function missense mutation is in the amino acid V534.

Embodiment 130. The method of embodiment 129, wherein the mutation is V534E.

Embodiment 131. The method of embodiment 116, wherein the at least one gain of function missense mutation is in the amino acid 5463.

Embodiment 132. The method of embodiment 131, wherein the mutation is S463P.

Embodiment 133. The method of embodiment 116, wherein the at least one gain of function missense mutation is in the amino acid V392.

Embodiment 134. The method of embodiment 133, wherein the mutation is V3921.

Embodiment 135. The method of embodiment 116, wherein the at least one gain of function missense mutation is in the amino acid E380.

Embodiment 136. The method of embodiment 135, wherein the mutation is E380Q.

Embodiment 137. The method of embodiment 100, wherein the ER⁺ breast cancer is visceral metastatic.

Embodiment 138. The method of any one of embodiments 96-137, wherein the breast cancer had progressed on one or more prior endocrine therapies.

Embodiment 139. The method of embodiment 138, wherein the prior endocrine therapy is a selective ER degrader (SERD), a selective ER modulator (SERM), optionally a SERM other than lasofoxifene, an aromatase inhibitor (AI), a mTOR inhibitor, and/or a PI3K inhibitor.

Embodiment 140. The method of embodiment 139, wherein the SERD is fulvestrant.

Embodiment 141. The method of embodiment 139, wherein the mTOR inhibitor is selected from sirolimus, temsirolimus, everolimus, or ridaforolimus.

Embodiment 142. The method of embodiment 141, wherein the mTOR inhibitor is everolimus.

Embodiment 143. The method of any one of embodiments 96-142, wherein the patient derives clinical benefit with stable disease for at least 24 weeks, 28 weeks, 32 weeks, 36 weeks, weeks, 44 weeks, 48 weeks, 52 weeks, 56 weeks, 60 weeks, 64 weeks, 68 weeks, 72 weeks, 74 weeks, 78 weeks, 82 weeks, 86 weeks, or longer following commencement of treatment with lasofoxifene or a pharmaceutically acceptable salt thereof and a CDK4/6i.

Embodiment 144. The method of embodiment 143, wherein the patient has complete or partial response to the treatment with lasofoxifene or a pharmaceutically acceptable salt thereof and a CDK4/6i.

Embodiment 145. The method of any one of embodiments 96-144, wherein the patient derives a longer duration of stable disease on treatment with lasofoxifene or a pharmaceutically acceptable salt thereof and a CDK4/6i than on the patient's preceding second-line or third-line therapies by at least about 20%, 15%, or 10%.

Embodiment 146. A method of monitoring a patient on a breast cancer treatment, comprising:
(a) determining quantitative measures of the mutant allele frequency (MAF) in circulating tumor DNA (ctDNA) of at least one gain of function missense mutation within the ligand binding domain (LBD) of the Estrogen Receptor 1 (ESR1) gene (ESR1 ctDNA) in a biological sample of the patient, wherein the quantitative measures are performed over a period of time at determined intervals; and
(b) determining a positive predictive value (PPV) for clinical benefit with stable disease of the cancer treatment,
wherein the PPV indicates responsiveness to the cancer treatment, and
wherein the cancer treatment comprises an effective amount of lasofoxifene or a pharmaceutically acceptable salt thereof and an effective amount of a CDK4/6 inhibitor (CDK4/6i).

Embodiment 147. The method of embodiment 146, wherein the patient has decreased MAF post treatment.

Embodiment 148. The method of any one of embodiments 146-147, wherein the quantitative measures of MAF are determined at 0 week, 4 weeks, 8 weeks, 12 weeks, 16 weeks, weeks, and/or 24 weeks.

Embodiment 149. The method of embodiment 148, wherein the quantitative measures of MAF are determined at every 4 weeks.

Embodiment 150. The method of any one of embodiments 146-149, wherein the patient derives clinical benefit with stable disease for at least 24 weeks, 28 weeks, 32 weeks, 36 weeks, 40 weeks, 44 weeks, 48 weeks, 52 weeks, 56 weeks, 60 weeks, 64 weeks, 68 weeks, 72 weeks, 74 weeks, 78 weeks, 82 weeks, 86 weeks, or longer.

Embodiment 151. The method of any one of embodiments 146-150, wherein the biological sample is blood, plasma, or serum.

Embodiment 152. The method of any one of embodiments 146-151, further comprising the earlier step of: determining that the patient has at least one gain of function missense mutation within the ligand binding domain (LBD) of the Estrogen Receptor 1 (ESR1) gene.

Embodiment 153. The method of embodiment 152, wherein the at least one of gain of function missense mutation is in any one of amino acids D538, Y537, L469, L536, P535, V534, S463, V392, and E380.

Embodiment 154. The method of embodiment 153, wherein the at least one gain of function missense mutation is in amino acid D538.

Embodiment 155. The method of embodiment 154, wherein the mutation is D538G.

Embodiment 156. The method of embodiment 152, wherein the at least one gain of function missense mutation is in the amino acid Y537.

Embodiment 157. The method of embodiment 152, wherein the mutation is Y537S, Y537N, Y537C, or Y537Q.

Embodiment 158. The method of embodiment 157, wherein the mutation is Y537C.

Embodiment 159. The method of embodiment 157, wherein the mutation is Y537S.

Embodiment 160. The method of embodiment 152, wherein the at least one gain of

function missense mutation is in the amino acid L469.

Embodiment 161. The method of embodiment 160, wherein the mutation is L469V.

Embodiment 162. The method of embodiment 152, wherein the at least one gain of function missense mutation is in the amino acid L536.

Embodiment 163. The method of embodiment 162, wherein the mutation is L536R or L536Q.

Embodiment 164. The method of embodiment 152, wherein the at least one gain of function missense mutation is in the amino acid P535.

Embodiment 165. The method of embodiment 164, wherein the mutation is P535H.

Embodiment 166. The method of embodiment 152, wherein the at least one gain of function missense mutation is in the amino acid V534.

Embodiment 167. The method of embodiment 166, wherein the mutation is V534E.

Embodiment 168. The method of embodiment 152, wherein the at least one gain of function missense mutation is in the amino acid S463.

Embodiment 169. The method of embodiment 168, wherein the mutation is S463P.

Embodiment 170. The method of embodiment 152, wherein the at least one gain of function missense mutation is in the amino acid V392.

Embodiment 171. The method of embodiment 170, wherein the mutation is V3921.

Embodiment 172. The method of embodiment 152, wherein the at least one gain of function missense mutation is in the amino acid E380.

Embodiment 173. The method of embodiment 172, wherein the mutation is E380Q.

Embodiment 174. The method of any one of embodiments 146-173, wherein the breast cancer is ER⁺ breast cancer.

Embodiment 175. The method of embodiment 174, wherein the ER⁺ breast cancer is HER2⁻.

Embodiment 176. The method of embodiment 174 or 175, wherein the ER⁺ breast cancer is locally advanced.

Embodiment 177. The method of embodiment 174 or 175, wherein the ER⁺ breast cancer is metastatic.

Embodiment 178. The method of any one of embodiments 146-177, wherein lasofoxifene is administered as lasofoxifene tartrate.

Embodiment 179. The method of any one of embodiments 146-178, wherein lasofoxifene is administered at 5 mg/day per os.

Embodiment 180. The method of any one of embodiments 146-179, wherein the CDK4/6i administered to the patient is selected from palbociclib, ribociclib, and abemaciclib.

Embodiment 181. The method of embodiment 180, wherein the CDK4/6i administered to the patient is abemaciclib.

Embodiment 182. The method of embodiment 181, wherein abemaciclib is administered orally at 50 mg to 200 mg BID.

Embodiment 183. The method of embodiment 182, wherein abemaciclib is administered orally at 100 mg to 200 mg BID.

Embodiment 184. The method of embodiment 183, wherein abemaciclib is administered orally at 150 mg BID.

Embodiment 185. The method of any one of embodiments 146-184, wherein the subject has progressed during prior CDK4/6 inhibitor therapy.

Embodiment 186. The method of embodiment 185, wherein the CDK4/6i administered to the patient is selected from palbociclib, ribociclib, and abemaciclib.

Embodiment 187. The method of embodiment 186, wherein the CDK4/6i administered to the patient is abemaciclib.

Embodiment 188. The method of any one of embodiments 146-187, wherein the subject has an oncogenic mutation in one or more genes other than ESRD.

Embodiment 189. The method of embodiment 188, wherein at least one of the one or more genes with an oncogenic mutation is selected from HNF1A, TERT, GATA3, CDK12, MAPK3, GNAS, RAF1, RHEB, NTRK3, TP53, PIK3CA, APC, ATM, MET, CCND1, EGFR, ROS1, BRCA2, STK11, FGFR1, ARID1A, CCNE1, AR, ALK, ERBB2, KIT, CDK4, SMAD4, NOTCH1, RB1, BRAF, PTEN, AKT1, CDH1, BRCA1, MYC, CDKN2A, IDH2, MTOR, or PDGFRA.

Embodiment 190. The method of embodiment 189, wherein each of the one or more genes with an oncogenic mutation is selected from HNF1A, TERT, GATA3, CDK12, MAPK3, GNAS, RAF1, RHEB, NTRK3, TP53, PIK3CA, APC, ATM, MET, CCND1, EGFR, ROS1, BRCA2, STK11, FGFR1, ARID1A, CCNE1, AR, ALK, ERBB2, KIT, CDK4, SMAD4, NOTCH1, RB1, BRAF, PTEN, AKT1, CDH1, BRCA1, MYC, CDKN2A, IDH2, MTOR, or PDGFRA.

Embodiment 191. The method of any one of embodiments 146-190, wherein the one or more genes with an oncogenic mutation is selected from HNF1A, TERT, GATA3, CDK12, MAPK3, TP53, PIK3CA, APC, ATM, MET, CCND1, EGFR, ROS1, STK11, FGFR1, ARID1A, CCNE1, AR, ALK, ERBB2, KIT, CDK4, SMAD4, NOTCH1, RB1, BRAF, RAF1, PTEN, AKT1, CDH1, BRCA1, MYC, CDKN2A, or PDGFRA.

Embodiment 192. The method of any one of embodiments 146-190, wherein the one or more genes with an oncogenic mutation is selected from CCND1, FGFR1, CCNE1, AR, ALK, MAPK3, KIT, SMAD4, NOTCH1, RB1, BRAF, RAF1, PTEN, AKT1, CDH1, BRCA1, MYC, CDKN2A, or PDGFRA.

Embodiment 193. The method of any one of embodiments 146-190, wherein the one or more genes with an oncogenic mutation is selected from HNF1A, TERT, GATA3, CDK12, MAPK3, TP53, PIK3CA, APC, ATM, MET, CCND1, EGFR, ROS1, BRCA2, STK11, FGFR1, ARID1A, CCNE1, AR, ALK, ERBB2, KIT, BRAF, or CDK4.

Embodiment 194. The method of embodiment 193, wherein the one or more genes with an oncogenic mutation is selected from TP53, PIK3CA, CCND1, ARID1A, FGFR1, CCNE1 and ERBB2.

Embodiment 195. The method of embodiment 194, wherein the oncogenic mutation is PIK3 CA.

Embodiment 196. The method of any one of embodiments 146-190, wherein the one or more genes with an oncogenic mutation is selected from HNF1A, TERT, GATA3, CDK12, MAPK3, GNAS, RAF1, RHEB, or NTRK3.

Embodiment 197. The method of any one of embodiments 146-196, wherein the subject has increased expression of one or more genes other than ESR1.

Embodiment 198. The method of embodiment 197, wherein the at least one of the one or more genes with an increased expression is selected from ABL1, AKT1, AKT2, ALK, APC, AR, ARID1A, ASXL1, ATM, AURKA, BAP, BAP1, BCL2L11, BCR, BRAF, BRCA1, BRCA2, CCND1, CCND2, CCND3, CCNE1, CDH1, CDK4, CDK6, CDK8, CDKN1A, CDKN1B, CDKN2A, CDKN2B, CEBPA, CTNNB1, DDR2, DNMT3A, E2F3, EGFR, EML4, EPHB2, ERBB2, ERBB3, ESR1, EWSR1, FBXW7, FGF4, FGFR1, FGFR2, FGFR3, FLT3, FRS2, HIF1A, HRAS, IDH1, IDH2, IGF1R, JAK2, KDM6A, KDR, KIFSB, KIT, KRAS, LRP1B, MAP2K1, MAP2K4, MCL1, MDM2, MDM4, MET, MGMT, MLL, MPL, MSH6, MTOR, MYC, NF1, NF2, NKX2-1, NOTCH1, NPM, NRAS, PDGFRA, PIK3CA, PIK3R1, PML, PTEN, PTPRD, RARA, RB1, RET, RICTOR, ROS1, RPTOR, RUNX1, SMAD4, SMARCA4, SOX2, STK11, TET2, TP53, TSC1, TSC2, or VHL.

Embodiment 199. The method of embodiment 198, wherein each of the one or more genes with an increased expression is selected from ABL1, AKT1, AKT2, ALK, APC, AR, ARID1A, ASXL1, ATM, AURKA, BAP, BAP1, BCL2L11, BCR, BRAF, BRCA1, BRCA2, CCND1, CCND2, CCND3, CCNE1, CDH1, CDK4, CDK6, CDK8, CDKN1A, CDKN1B, CDKN2A, CDKN2B, CEBPA, CTNNB1, DDR2, DNMT3A, E2F3, EGFR, EML4, EPHB2, ERBB2, ERBB3, ESR1, EWSR1, FBXW7, FGF4, FGFR1, FGFR2, FGFR3, FLT3, FRS2, HIF1A, HRAS, IDH1, IDH2, IGF1R, JAK2, KDM6A, KDR, KIF5B, KIT, KRAS, LRP1B, MAP2K1, MAP2K4, MCL1, MDM2, MDM4, MET, MGMT, MLL, MPL, MSH6, MTOR, MYC, NF1, NF2, NKX2-1, NOTCH1, NPM, NRAS, PDGFRA, PIK3CA, PIK3R1, PML, PTEN, PTPRD, RARA, RB1, RET, RICTOR, ROS1, RPTOR, RUNX1, SMAD4, SMARCA4, SOX2, STK11, TET2, TP53, TSC1, TSC2, or VHL.

Embodiment 200. The method of any one of embodiments 197-199, wherein the one or more genes with an increased expression is selected from AKT1, AKT2, BRAF, CDK4, CDK6, PIK3CA, PIK3R1, or mTOR.

Embodiment 201. The method of embodiment 177, wherein the ER⁺ breast cancer is visceral metastatic.

Embodiment 202. The method of any one of embodiments 146-201, wherein the breast cancer had progressed on one or more prior endocrine therapies.

Embodiment 203. The method of embodiment 202, wherein the prior endocrine therapy is a selective ER degrader (SERD), a selective ER modulator (SERM), an aromatase inhibitor (AI), a mTOR inhibitor, and/or a PI3K inhibitor.

Embodiment 204. The method of embodiment 203, wherein the SERD is fulvestrant.

Embodiment 205. The method of embodiment 203, wherein the mTOR inhibitor is selected from sirolimus, temsirolimus, everolimus, and ridaforolimus.

Embodiment 206. The method of embodiment 205, wherein the mTOR inhibitor is everolimus.

### 6. EXAMPLES

### 6.1. Example 1: Phase 2 Clinical Study (ELAINE 2)

A phase 2 clinical study, NCT04432454 (ELAINE 2), is currently underway to evaluate the efficacy, safety and tolerability of the combination of lasofoxifene and abemaciclib for the treatment of postmenopausal women and certain premenopausal women with locally advanced or metastatic estrogen receptor positive (ER⁺)/human epidermal growth factor 2 negative (HER2⁻) (ER⁺/HER2⁻) breast cancer having an ESR1 mutation. As discussed in Section 5.1.1 below, the study has enrolled patients whose tumors had progressed on prior CDK4/6 inhibitor treatment.

### 6.1.1. Summary of Experimental Observations

ELAINE 2 is an open-label, phase 2, multicenter trial evaluating the safety and efficacy of LAS combined with the CDK4/6i, abemaciclib (Abema). Study participants were pre- and postmenopausal women with ER⁺/HER2⁻ mBC (metastatic breast cancer) with acquired ESR1 mutation (identified by ctDNA testing), whose disease had progressed on one or two lines of hormonal therapy for metastatic disease with or without a CDK4/6i (including Abema). Patients took oral lasofoxifene (LAS) 5 mg/day and abemaciclib 150 mg BID. Treatment continued until evidence of disease progression, death, unacceptable toxicity, or withdrawal from the study. The primary endpoint was safety, and secondary endpoints were progression free survival (PFS), objective response rate (ORR), and clinical benefit rate (CBR).

Twenty-nine (29) patients were enrolled at 16 US sites (October 2020 to June 2021). Mean age was 58.3 y (35-79 y); 86% were Caucasian. Most had progressed with at least 2 previous hormonal treatments (80%). All except 1 patient had received a prior CDK4/6i and 72% had received prior fulvestrant (FVT); 48% had had chemotherapy in the metastatic setting. Four patients discontinued the trial due to adverse events (AEs, n=2), consent withdrawal (n=1), or investigator withdrawal (n=1). No deaths occurred during the study and few Grade 3/4 AEs were observed. Most common AEs were diarrhea, nausea, and leukopenia. Five patients had an abemaciclib dose reduction from 150 mg to 100 mg BID. As of the first interim date, 11 patients have progressed and 14 continue treatment. The censored median PFS was 13.9 mos (95% CI, 8.0-NE), the ORR 33.3% (95% CI, 16.3-56.3) with 6 confirmed partial responses, and the CBR 62.1% (95% CI, 44.0-77.3).

As shown in FIGS. 1-4, patients derived clinical benefit (CB, defined as stable disease ≥24 weeks, or confirmed partial or complete response) from lasofoxifene in combination with abemaciclib even after progression on prior cyclin-dependent kinase 4/6 inhibitor (CDK4/6i) treatment, including prior treatment with ribociclib, palbociclib, and/or abemaciclib.

Conclusion: lasofoxifene combined with abemaciclib was well tolerated and demonstrated robust and meaningful efficacy in women with ER⁺/HER2⁻ metastatic breast cancer (mBC) and an ESR1 mutation who had progressed on previous CDK4/6i therapies.

### 6.1.2. Study Design

This is an open-label, multicenter, study evaluating the efficacy, safety and tolerability of the combination of lasofoxifene and abemaciclib for the treatment of pre- and postmenopausal women who have locally advanced or metastatic ER⁺/HER2⁻ breast cancer with an ESR1 mutation and disease progression on first, second or third lines of hormonal treatment for metastatic disease. To be eligible for enrollment, progression may have occurred on no more than three of the following treatments for metastatic breast cancer: an AI and/or fulvestrant, either as monotherapy or in combination with any commercially available CDK4/6i; and/or the combination of fulvestrant and alpelisib; and/or tamoxifen; and/or the combination of exemestane/everolimus.

On the day of enrollment (Day 1), subjects received 5 mg of oral lasofoxifene once a day and 150 mg of abemaciclib twice a day. Study medication is continued until documented breast cancer progression or withdrawal from the study for any reason.

Treatment continues until radiographic or clinical evidence of disease progression, death, unacceptable toxicity, or until withdrawal from the study for any reason. Enrolled subjects are seen every 2 weeks for the first two months of treatment and then monthly until progression. Efficacy assessments are done every 8 weeks. Safety assessments are done at weeks 2, 4, 6, and 8 after enrollment and then every month until disease progression.

### 6.1.3. Drug Schedules

Subjects receive oral lasofoxifene 5 mg once a day (one tablet) and oral abemaciclib 150 mg (3 tablets) twice daily with or without food. Study medication will continue to be given until documented breast cancer progression or withdrawal from the study for any reason.

### Lasofoxifene

The active pharmaceutical ingredient is lasofoxifene. The chemical name is 65-Phenyl-5R-[4-(2-pyrrolidin-1-yl-ethoxy)-phenyl]-5,6,7,8-tetrahydro-naphthalen-ol,2S,3 S-Dihyroxy-succinic acid.

### Chemical Structure

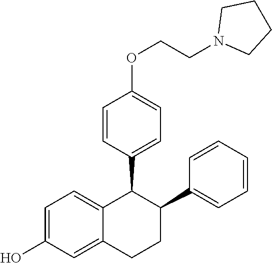 [SMILES: Oc1ccc2c(c1)CC[C@H](c1ccccc1)[C@@H]2c1ccc(OCCN2CCCC2)cc1]

Molecular formula: C₂₈H₃₁NO₂·C₄H₆O₆
Molecular weight: 563.64 Daltons
Physical appearance: white to off-white solid
Lasofoxifene is provided as a white to off-white solid 5 mg tablet using the D-(-)-tartrate salt.

### Abemaciclib

Abemaciclib is a kinase inhibitor and the chemical name is 2-Pyrimidinamine, N-[5-[(4-ethyl-1-piperazinyl)methyl]-2-pyridinyl]-5-fluoro-4-[4-fluoro-2-methyl-1-(1-methylethyl)-1H-b enzimidazol-6-yl .

Molecular formula: C27H32F2N8
Molecular weight: 506.59 Daltons
Physical appearance: white to yellow powder

### 6.1.3.1 Dose Modifications for Treatment Toxicity

### Lasofoxifene

There are no dose reductions for any toxicity associated with lasofoxifene. If a lasofoxifene related Grade 3 or 4 AE event occurs, hold therapy until the toxicity resolves to <Grade 2 or baseline and resume lasofoxifene at the assigned dose. Should a patient need to be NPO, hold lasofoxifene and abemaciclib until the subject can take fluids by mouth. If a subject cannot tolerate lasofoxifene or has not taken lasofoxifene for 3 consecutive weeks, the subject should be withdrawn from the study.

### Physical Appearance: White to Yellow Powder

### Abemaciclib

Two abemaciclib dose modifications for adverse reactions are permitted:
Starting dose—150 mg twice a day (3 tablets twice a day)
First dose reduction—100 mg twice a day (2 tablets twice a day)
Second dose reduction—50 mg twice a day (1 tablet twice a day)
Third dose reduction—not allowed
   If subjects are unable to tolerate 50 mg of abemaciclib twice daily, this treatment should be stopped; however, lasofoxifene can be continued until disease progression is documented. However, if lasofoxifene is discontinued, abemaciclib cannot be continued as monotherapy and the subject needs to be withdrawn from the study. Dose modifications for treatment toxicity are summarize in Table 1 below.

TABLE 1
Criteria for abemaciclib dose reductions

| CTCAE Grade | Hematological |
|---|---|
| Grade 1 & 2 | No dose modification is required |
| Grade 3 | Suspend dose until toxicity resolves to ≤Grade 2; Dose |
| | reduction is not required |
| Grade 3 recurrent, or Grade 4 | Suspend dose until toxicity resolves to ≤Grade 2; Resume |
| | dosing at the next lower dose |
| CTCAE Grade | Diarrhea |
| Grade 1 | No dose modification is required |
| Grade 2 | If toxicity does not resolve within 24 hours to ≤Grade 1, |
| Grade 2 that persists or recurs after | suspend dose until toxicity resolves. |
| resuming the same dose despite | No dose reduction |
| maximal supportive measures | Suspend dose until toxicity resolves to ≤Grade 1 Resume at |
| Grade 3 or 4 or requires hospitalization | next lower dose |
| | Suspend dose until toxicity resolves to ≤Grade 1 Resume at |
| | next lower dose |
| CTCAE Grade for ALT and AST | Hepatotoxicity |
| Grade 1 (>ULN-3.0 × ULN) | No dose modification is required |
| Grade 2 (>3.0-5.0 × ULN), WITHOUT | |
| increase in total bilirubin above 2 × | |
| ULN | |
| Persistent or Recurrent Grade 2 or | Suspend dose until toxicity resolves to baseline or Grade 1, |
| Grade 3 | Resume at next lower dose. |
| (>5.0-20.0 × ULN), WITHOUT | |
| increase in total bilirubin above 2 × | |
| ULN | |
| Elevation in AST and/or ALT >3 × | Discontinue abemaciclib |
| ULN WITH total bilirubin >2 × ULN, | |
| in the absence of cholestasis | |
| Grade 4 (>20.0 × ULN) | Discontinue abemaciclib |
| CTCAE Grade | Interstitial lung disease/pneumonitis |
| Grade 1 or 2 | No dose modification is required |
| Persistent or recurrent Grade 2 toxicity | Suspend dose until toxicity resolves to ≤Grade 1 |
| that does not resolve with maximal | Resume at next lower dose |
| supportive measures within 7 days to | |
| baseline or Grade 1 | |
| Grade 3 or 4 | Discontinue abemaciclib |
| CTCAE Grade | Other Toxicities |
| Grade 1 or 2 | No dose modification is required |
| Persistent or recurrent Grade 2 toxicity | Suspend dose until toxicity resolves to baseline or ≤Grade 1. |
| that does not resolve with maximal | Resume at next lower dose |
| supportive measures within 7 days to | |
| baseline or Grade 1 | |
| Grade 3 or 4 | Suspend dose until toxicity resolves to baseline or ≤Grade 1. |
| | Resume at next lower dose |

### 6.1.4. Primary and Secondary Endpoints

The primary endpoint is evaluation of the safety and tolerability of the combination of lasofoxifene and abemaciclib for the treatment of postmenopausal women with locally advanced or metastatic ER⁺/HER2⁻ breast cancer and have an ESR1 mutation based on the incidence of adverse events, severity of the adverse events and mortality due to adverse events. The pharmacokinetic endpoint is to determine if there are any drug-drug interactions between lasofoxifene and abemaciclib as compared with steady-state drug concentrations obtained in previous clinical trials.

The secondary endpoints include: progression free survival (PFS); clinical benefit (CB, defined as stable disease ≥24 weeks, or confirmed partial or complete response) rate (CBR), duration of response, objective response rate (ORR), quality of life (QoL), and time to response. For subjects with measurable disease at baseline, progression is determined according to the RECIST criteria.

### 6.1.5. Inclusion and Exclusion Criteria

The inclusion criteria include:
1. Pre- or postmenopausal.
   Postmenopausal women are defined as:
a. ≥60 years of age with no vaginal bleeding over the prior year, or
b. <60 years with “premature menopause” or “premature ovarian failure” manifest itself with secondary amenorrhea for at least 1 year and follicle stimulating hormone (FSH) and estradiol levels in the postmenopausal range according to institutional standards, or
c. surgical menopause with bilateral oophorectomy.
   Note: Premenopausal women who meet all of the other entry criteria must be maintained on ovarian suppression (such as Lupron) during the study and subjects counseled to use appropriate contraception to prevent pregnancy.
2. If possible, a biopsy of metastatic breast cancer tissue will be obtained to provide histological or cytological confirmation of ER+ and HER2− disease as assessed by a local laboratory, according to American Society of Clinical Oncology/College of American Pathologists guidelines, using slides, paraffin blocks, or paraffin samples. If a biopsy is not possible, the ER and HER2 status from the tissue obtained at the time of the original diagnosis must confirm that the subject is ER+ and HER2−.
3. Locally advanced or metastatic breast cancer with radiological or clinical evidence of progression on first and/or second line of hormonal therapy for metastatic disease. Progression may have occurred on no more than 2 of the following endocrine treatments for metastatic breast cancer: an aromatase inhibitor (AI) and/or fulvestrant either as monotherapy or in combination with any commercially approved CDK4/6i; and/or the combination of fulvestrant and alpelisib; and/or tamoxifen; and/or the combination of exemestane/everolimus. (Note: Before starting study treatment, subjects should have stopped any CDK4/6i for at least 21 days)
4. Subjects must have had no evidence of progression for at least 6 months during their first hormonal treatment for advanced breast cancer.
5. At least one or more of the following ESR1 point mutations as assessed in cell-free circulating tumor DNA (ctDNA) obtained from a blood or tissue sample: Y537S, Y537C, D538G, E380Q, S463P, V534E, P535H, L536H, L536P, L536R, L536Q, or Y537N. Note: the Sponsor's blood ctDNA assay must be used but tissue sequencing (if done) may be done by a validated commercial assay.
   Note: A positive ESR1 mutation in tissue or ctDNA using a validated commercial assay if done prior or at the time of disease progression is acceptable to meet this entry criteria. However, blood for ctDNA must still be obtained for genomic analyses using the sponsor's ctDNA assay.
6. Locally advanced or metastatic breast cancer with either measurable (according to RECIST 1.1 [Eisenhauer et al. New response evaluation criteria in solid tumours: Revised RECIST guideline (version 1.1). European Journal of Cancer. 2009; 45:228-47.]) or non-measurable lesions.
7. Subjects who may have received one cytotoxic chemotherapy regime for metastatic disease, as well as those who received one cytotoxic chemotherapy regimen in the neo-adjuvant or adjuvant setting prior to entry into the trial, can be enrolled but must be free of all chemotherapy acute toxicity, excluding alopecia and Grade 2 peripheral neuropathy, before study entry. A washout period of at least 21 days is required between last chemotherapy dose and entry into the study.
8. Stable breast cancer metastasis to the brain is allowed as long as the subject has received radiotherapy and not demonstrated any evidence of brain metastasis progression for at least 3 months after the completion of radiotherapy.
9. ECOG performance score of 0 or 1.
10. Adequate organ function as shown by:
a. absolute neutrophil count (ANC) ≥1,500 cells/mm³
b. platelet count ≥100,000 cells/mm 3
c. hemoglobin ≥8.0 g/dl
d. ALT and AST levels ≤3 upper limit of normal (ULN) or ≤5 in the presence of liver metastasis
e. total serum bilirubin ≤0.5×ULN (≤3.0×ULN for subjects known to have Gilbert Syndrome)
f. alkaline phosphatase level ≤3×ULN
g. creatinine clearance of 40 ml/min or greater as calculated by the Cockcroft-Gault formula
h. international normalized ratio (INR) and activated partial thromboplastin time (aPTT) <2.0×ULN
11. Able to swallow tablets.
12. Able to understand and voluntarily sign a written informed consent before any screening procedures.

Exclusion criteria. Subjects who meet any of the following criteria are excluded from entering the trial.
1. Lymphangitic carcinomatosis involving the lung.
2. Visceral crisis in need of cytotoxic chemotherapy as assessed by the investigator.
3. Radiotherapy within 30 days prior to entry into the trial except in case of localized radiotherapy for analgesic purposes or for lytic lesions at risk of fracture, which can then be completed within 7 days prior to entry into the trial. Subjects must have recovered from radiotherapy toxicities prior entry.
4. Subjects with known inactivating RB1 mutations or deletions (Screening for RB1 mutation is not required for entry).
5. History of long QTC syndrome or a QTC of >480 msec.
6. History of a pulmonary embolus (PE) or deep vein thrombosis (DVT) within the last 6 months or any known thrombophilia. Subjects stable on anti-coagulants for maintenance are eligible as long as the DVT and/or PE occurred >6 months prior to enrollment and there is no evidence for active thrombosis. The use of low-dose ASA is permitted.
7. Subjects on concomitant strong CYP3A4 inhibitors such as clarithromycin, telithromycin, nefazodone, itraconazole, ketoconazole, atazanavir, darunavir, indinavir, lopinavir, nelfinavir, ritonavir, saquinavir, tipranavir.
8. Subjects on strong and moderate CYP3A4 inducers such as amprenavir, barbituates, carbamazepine, clotrimazole, dexamethasone, efavirenz, ethosuximide, griseofulvin, modafinil, nevirapine, oxcarbazepine, phenobarbital, phenytoin, chronic prednisone treatment, primidone, rifabutin, rifampin, rifapentine, ritonavir, topiramate.
9. Any significant co-morbidity that would impact the study or the subject's safety. Since CDK4/6i have reported the occurrence of interstitial lung disease (ILD), subjects who have a history of ILD or have severe dyspnea at rest or require oxygen therapy should not enter the study.
10. Subject has an active systemic bacterial or fungal infection (requiring intravenous [IV] antibiotics at the time of initiating treatment).
11. History of a positive human immunodeficiency virus (HIV) or hepatitis B virus (HBV) test [Screening is not required for enrollment].
12. Subjects with hepatitis C virus (HCV) at Screening who still have a viral load. Subjects previously treated and achieved a HCV cure (no viral load) can be entered into the study.
13. History of malignancy within the past 5 years (excluding breast cancer), except basal cell or squamous cell carcinoma of the skin curatively treated by surgery, or early-stage cervical cancer.
14. A positive pregnancy test (only if premenopausal).
15. History of non-compliance to medical regimens.
16. Unwilling or unable to comply with the protocol.
17. Current participation in any clinical research trial involving an investigational drug or device within the last 30 days.

### 6.1.6. Efficacy Analysis

A Kaplan-Meier curve is presented for PFS with an estimated median PFS. The clinical benefit rate (CBR), defined as the percentage of subjects with a complete or partial response or stable disease for ≥24 weeks, is presented with a 95% confidence interval. The ORR, defined as the percentage of subjects with a complete or partial response, is similarly summarized. Time to response and duration of response (DoR) is presented for each individual responder.

### 6.1.7. Safety Analysis

For the Safety Population, descriptive summaries of AEs, clinical laboratory data, vital signs, and ECGs are presented.

Verbatim descriptions of AEs reported during the study period are mapped to the appropriate system organ class and preferred term using the Medical Dictionary for Regulatory Activities (MedDRA). All reported AEs are tabulated and graded according to the CTCAE version 5.0. AEs are summarized by worst grade per subject and by grade. All treatment-emergent adverse events (TEAEs) (i.e., occurring during or after the first dose of study drug) are summarized in frequency tables. Treatment-emergent serious AEs and TEAEs that resulted in early study discontinuation are listed and summarized in frequency tables. The numbers and proportions of subjects who discontinue either or both study treatments early due to AEs are reported with 95% confidence intervals. Subjects experiencing key adverse events of interest are similarly summarized. With 24 evaluable subjects, the upper half width of the Wilson score 95% confidence interval is estimated to within 20%. An adverse event with a 7% incidence rate has a chance of being seen at least once.

All AEs that led to death are listed by subject with narratives.

Clinical laboratory test results are summarized using descriptive statistics for absolute values and change-from-baseline values summarized as cumulative shift tables.

### 6.1.8. Analysis of Pharmacokinetic Variables

Pharmacokinetic sampling for lasofoxifene and abemaciclib concentrations are be done pre-dose at every visit starting at Visit 0 (Day 1) through Final/ET visit. Pharmacokinetic concentrations of lasofoxifene, abemaciclib and 3 abemaciclib metabolites (LSN2839567, LSN3106726, and LSN3106729) are summarized and mean, median, SD, and range at each time are presented and compared with previous PK obtained results.

### 6.2. Example 2: First Interim Results of ELAINE 2 Clinical Trial

Twenty-nine (29) patients total were enrolled. Patient disposition is summarized in Table 2. Patient demography and baseline characteristics are summarized in Table 3, and patient prior cancer therapies are summarized in Table 4.

### 6.2.1. Patient Disposition

### 6.2.2. Demography and Baseline Characteristics n=29

TABLE 3

| | | N = 29 | |
|---|---|---|---|
| Age (yrs) | Mean | 58.3 | |
| | Median | 60 | |
| | Range | 35-79 | |
| Menopause | Pre n (%) | 4 | (13.8%) |
| | Post n (%) | 25 | (86.2%) |
| Ethnicity | Non-Hispanic | 26 | (89/7%) |
| | Hispanic | 3 | (10.3%) |
| Race | White | 25 | (86.2%) |
| | Black | 2 | (6.9%) |
| | Not Reported | 2 | (6.9%) |
| Measurable Disease | | 18 | (62.1%) |
| Visceral Disease | | 16 | (55.2%) |
| Bone Only | | 10 | (34.5%) |

### 6.2.3. Prior Breast Cancer Therapy

TABLE 4

| Prior breast cancer therapy | N = 29 | |
|---|---|---|
| Chemotherapy (total) | 25 | (86.2) |
| Chemotherapy in metastatic setting | 14 | (48.3) |
| CDK4/6i | 28 | (96.6) |
| Palbociclib | 25 | (86.2) |
| Abemaciclib | 4 | (13.8) |
| Ribociclib | 2 | (6.9) |
| Unknown | 1 | (3.4) |
| Endocrine therapy | 29 | (100) |
| Aromatase inhibitor | 28 | (96.6) |
| Fulvestrant | 23 | (79.3) |
| Tamoxifen | 12 | (41.4( |
| Everolimus | 4 | (13.8) |
| Alpelisib | 3 | (10.3) |

* Data expressed as n (%), unless stated otherwise. CDK4/6i, cyclin-dependent kinase 4/6 inhibitor. 26 (89.7%) patients had prior radiotherapy.

### 6.2.4. ELAINE 2 SwimmerP lot

Patient response to lasofoxifene and CDK 4/6 inhibitor therapy as of a first interim date is summarized in FIG. 1.

Referring to FIG. 1, as of the first interim date, about 68.9% (20/29) of subjects receiving lasofoxifene and abemaciclib (laso/abema) treatment had clinical benefit (CB) with stable disease and showed complete or partial response for at least 24 weeks (vertical dotted line) post treatment. Among these patients, about 65% (13/20) had continued response (arrow) as of the first interim date. Patients 29, 21, 16, 6 (patients marked with an asterisk in FIG. 1) had had progression on abemaciclib prior to enrollment. Notably, 75% (3/4) of these patients had CB and stable disease to 40 weeks (patient 16), 48 weeks (patient 21), and 68 weeks (patient 29). Patients 16 and 6 progressed and withdrew at 40 weeks and 8 weeks, respectively.

Approximately 48% (14/29) of patients had progressed on prior, pre-enrollment, fulvestrant treatment (square). These patients generally responded well to laso/abema treatment. Approximately 64% (9/14) achieved CB and had complete or partial response to laso/abema treatment for up to 62 weeks as of the first interim date. For example, Patients 28, 27, 24, 19, and 14 had stable disease and continued response to laso/abema (arrow) to 62 weeks, 58 weeks, 54 weeks, 50 weeks, 42 weeks, and 38 weeks, respectively. Patients 15 and 13 had stable disease and progressed at 36 weeks and 32 weeks, respectively.

All patients had at least one missense mutation in ESR1 at enrollment. Approximately 69% (20/29) of patients had a Y537S mutation, and approximately 65% (13/20) of these patients achieved CB, and had complete or partial response and stable disease to 62 weeks. Patients 28, 27, 25, 24, 23, 21, 20, 19, 18, 17, 11, and 10 had stable disease and continued response to 62 weeks, 58 weeks, 54 weeks, 48 weeks, 48 weeks, 48 weeks, 48 weeks, 40 weeks, 40 weeks, 40 weeks, 32 weeks, and 32 weeks, respectively. Patients 15 and 13 had stable disease and progressed at 32 weeks.

Notably, 75% (12/16) of patients with visceral metastasis benefitted from laso/abema treatment and achieved CB, even if their cancers had metastasized to visceral organs prior to enrollment.

### 6.2.5. Progression Free Survival

Abemaciclib/Laso combination. Table 5 summarizes patient progression free survival (PFS).

TABLE 5

| | | PFS |
|---|---|---|
| # of Progression Events | Progression | 11 |
| | Death | 0 |
| Kaplan Meier PFS | Median | 56.0 weeks (13 mos) |
| | 95% CI | 31.9-NE |

### 6.2.6. Tumor Response Waterfall Plot

Preliminary data of maximum tumor response evaluated as of the first interim date is illustrated in FIG. 2. Patients having complete or partial response to laso/abema treatment had maximum % change in the sum of dimensions of target lesion of up to 80%. Nine (9/18) patients with measurable lesions had a partial response yielding an objective response rate (ORR) of 50% (95% confidence, 29.0-71.0). Table 6 summarizes patient ORR (ORR=50%), and Table 7 summarizes patient DOR and TTR.

### 6.2.7. ORR, DOR and TTR

Table 6 summarizes patient ORR (ORR=50%), and Table 7 summarizes patient DOR.

TABLE 6

| | N = 18¹ | 95% CI |
|---|---|---|
| CR | 0 (0%) | |
| PR | 9 (50%) | 12.5-50.9 |

TABLE 7

| | Mean ± SD | Median | Min-Max | No Censored |
|---|---|---|---|---|
| DOR² | 134.8 ± 49.97 | 164 | 57-173 | 2 |
| (Days) | | | | |
| TTR³ | 92.8 ± 53.60 | 69 | 54-176 | 0 |
| (Days | | | | |

### 6.2.8. Clinical Benefit Rate (CBR) N=29

Table 8 summarizes patient clinical benefit rate (CBR).

TABLE 8

| | Data as of |
|---|---|
| | first interim date |
| CR (n) | 0 |
| PR (n) | 9 |
| ≥24 wks (n) | 11* |
| CBR (%) | 69% |

*Number with SD excluding those with PR

### 6.2.9. Number of Subjects with Most Common Adverse Events (AEs)

Subjects with maximum Grade counts are summarized in Table 9.

TABLE 9

| | Grade 1 | | Grade 2 | | Grade 3 | | Grade 4 | |
|---|---|---|---|---|---|---|---|---|
| Adverse Events | # | | # | | # | | # | |
| by Grade | Subjects | % | Subjects | # | Subjects | # | Subjects | # |

| | | | | | | | |
|---|---|---|---|---|---|---|---|
| Diarrhea | 20 | 69.0% | 4 | 13.8% | | 0.0% | 0.00% |
| Nausea | 10 | 34.5% | 4 | 13.8% | | 0.0% | 0.00% |
| Fatigue | 6 | 20.7% | 3 | 10.3% | 1 | 3.4% | 0.00% |
| WBC Decreased | 3 | 10.3% | 6 | 20.7% | | 0.0% | 0.00% |
| Vomiting | 5 | 17.2% | 2 | 6.9% | 1 | 3.4% | 0.00% |
| Dyspnea | 4 | 13.8% | 2 | 6.9% | | 0.0% | 0.00% |
| Muscle Spasm | 6 | 20.7% | | 0.0% | | 0.0% | 0.00% |
| Lymph Decreased | 1 | 3.4% | 2 | 6.9% | 3 | 10.3% | 0.00% |
| Anemia | 4 | 13.8% | 1 | 3.4% | 1 | 3.4% | 0.00% |
| Myalgia | 5 | 17.2% | | 0.0% | | 0.0% | 0.00% |
| Cough | 4 | 13.8% | 1 | 3.4% | | 0.0% | 0.00% |
| Constipation | 5 | 17.2% | | 0.0% | | 0.0% | 0.00% |
| Dec appetite | 3 | 10.3% | 1 | 3.4% | | 0.0% | 0.00% |
| Hyperglycemia | 3 | 10.3% | 1 | 3.4% | | 0.0% | 0.00% |
| Hypokalemia | 1 | 3.4% | 1 | 3.4% | 2 | 6.9% | 0.00% |
| Dehydration | 2 | 6.9% | 2 | 6.9% | | 0.0% | 0.00% |
| Incr Creatnine | 3 | 1.3% | 2 | 6.9% | | 0.0% | 0.00% |

### 6.2.10. Number of Subjects with Hematologic Adverse Events (AEs)

Number of subjects with hematologic adverse events (AEs) are summarized in Table 10.

TABLE 10

| | Grade 1 | | Grade 2 | | Grade 3 | | Grade 4 | |
|---|---|---|---|---|---|---|---|---|
| Adverse Events | # | | # | | # | | # | |
| by Grade | Subjects | % | Subjects | # | Subjects | # | Subjects | # |
| Dec Neutrophils | 1 | 3.4% | 2 | 6.9% | | 0.0% | 1 | 3.4% |
| Neutropenia | | 0.0% | 0 | 0.0% | 1 | 3.4% | 1 | 3.4% |
| WBC Decreased | 3 | 10.3% | 6 | 20.7% | | 0.0% | | 0.0% |
| Lymph Decreased | 1 | 3.4% | 2 | 6.9% | 3 | 10.3% | | 0.0% |
| Anemia | 4 | 13.8% | 1 | 3.4% | 1 | 3.4% | | 0.0% |
| Dec PLts | 3 | 10.3% | | 0.0% | | 0.0% | | 0.0% |
| Thrombocytopenia | 1 | 3.4% | | 0.0% | | 0.0% | | 0.0% |
| Bone Marrow Failure | | 0.0% | | 0.0% | 1 | 3.4% | | 0.0% |

### 6.2.11. Number of Subjects with Liver Adverse Events (AEs)

Number of subjects with liver AEs are summarized in Table 11.

TABLE 11

| | Grade 1 | | Grade 2 | | Grade 3 | | Grade 4 | |
|---|---|---|---|---|---|---|---|---|
| Adverse Events | # | | # | | # | | # | |
| by Grade | Subjects | % | Subjects | # | Subjects | # | Subjects | # |
| Hypoalbuminemia | 4 | 13.8% | | 0.0% | | 0.0% | | 0.0% |
| AlkPhos Incr | 1 | 3.4% | 1 | 3.4% | | 0.0% | | 0.0% |
| Bilirubin Incr | 0 | 0.0% | 1 | 3.4% | | 0.0% | | 0.0% |
| ALT | 2 | 6.9% | | 0.0% | | 0.0% | | 0.0% |
| AST | 2 | 6.9% | | 0.0% | | 0.0% | | 0.0% |

### 6.2.12. Grade 3 and 4 Toxicities (n=29) Table 12 summarizes Grade 4 and 4 toxicities.

TABLE 12

| Adverse Events | Grade 3 - Severe | | Grade 4 - Life Threatening | |
|---|---|---|---|---|
| by SOC and Grade | # Subjects | % | # Subjects | # |
| Anemia | 1 | 3.4% | | 0.0% |
| Bone Marrow Failure | 1 | 3.4% | | 0.0% |
| Neutropenia | 1 | 3.4% | 1 | 3.4% |
| Neutrophil count decr | 0 | 0.0% | 1 | 3.4% |
| Lymph count decr | 3 | 10.3% | 1 | 3.4% |
| Hypokalemia | 2 | 6.9% | | 0.0% |
| Hyponatremia | 1 | 3.4% | | 0.0% |
| Hypoglycemia | 1 | 3.4% | | 0.0% |
| DVT/PE | 2 | 6.9% | | 0.0% |
| Vomiting | 1 | 3.4% | | 0.0% |
| Fatigue | 1 | 3.4% | | 0.0% |
| Fall | 1 | 3.4% | | 0.0% |
| Maculopapular rash | 1 | 3.4% | | 0.0% |
| Grand Total | 16 | | 3 | |

### 6.2.13. Adverse Events (AEs) of Special Interest

Table 13 summarizes adverse events (AEs) of special interest.

TABLE 13

| | Grade 1 | | Grade 2 | | Grade 3 | | Grade 4 | |
|---|---|---|---|---|---|---|---|---|
| Adverse Events | # | | # | | # | | # | |
| by Grade | Subjects | % | Subjects | # | Subjects | # | Subjects | # |
| Dec Neutrophils | 1 | 3.4% | 2 | 6.9% | | 0.0% | 1 | 3.4% |
| Neutropenia | | 0.0% | 0 | 0.0% | 1 | 3.4% | 1 | 3.4% |
| WBC Decreased | 3 | 10.3% | 6 | 20.7% | | 0.0% | | 0.0% |
| Lymph Decreased | 1 | 3.4% | 2 | 6.9% | 3 | 10.3% | | 0.0% |
| Anemia | 4 | 13.8% | 1 | 3.4% | 1 | 3.4% | | 0.0% |
| Dec PLts | 3 | 10.3% | | 0.0% | | 0.0% | | 0.0% |
| Thrombocytopenia | 1 | 3.4% | | 0.0% | | 0.0% | | 0.0% |
| Bone Marrow Failure | | 0.0% | | 0.0% | 1 | 3.4% | | 0.0% |
| Diarrhea | 20 | 69.0% | 4 | 13.8% | | 0.0% | | 0.0% |
| Fatigue | 6 | 20.7% | 3 | 10.3% | 1 | 3.4% | | 0.0% |
| Muscle Spasm | 6 | 20.7% | | 0.0% | | 0.0% | | 0.0% |
| Myalgia | 5 | 17.2% | | 0.0% | | 0.0% | | 0.0% |
| ALT | 2 | 6.9% | | 0.0% | | 0.0% | | 0.0% |
| AST | 2 | 6.9% | | 0.0% | | 0.0% | | 0.0% |
| Hot Flashes | 3 | 10.3% | 1 | 3.4% | | 0.0% | | 0.0% |
| DVT/PE | | 0.0% | | 0.0% | 2 | 6.9% | | 0.0% |
| UTI | | 0.0% | 3 | 10.3% | | 0.0% | | 0.0% |
| Vaginal Discharge | 1 | 3.4% | | 0.0% | | 0.0% | | 0.0% |

### 6.2.14. Summary of Dose Reduction

Lasofoxifene dose was not reduced per protocol
Abemaciclib
   No Patient was reduced to 50 mg BID
   Reduction from 150 mg BID to 100 mg BID
4 due to AE
   Hyponatremia
   Dizziness, Fatigue, Vomiting, Wt loss
   Inc Creatinine
   Anorexia, Fatigue, Nausea, Gen Muscle Weakness
1 Due to Investigator discretion

### 6.2.15. ELAINE 2 Interim PFS Data Comparison with Other Trials

As shown in Table 14 below, lasofoxifene in combination with abemaciclib provides greater median PFS compared to published and publicly presented data in patients who have had prior administration of CDk4/6i, and comparable efficacy to CDK4/6i- naïve patients; in greater detail, as compared to abema alone, either in CDK naïve patients (Monarch 1 trial) or in a post-CDK population (Abema trial); Piqray/fulvestrant in patients with PIK3CA mutations (Bylieve trial); camizestrant plus palbociclib (Serena-1 trial), amcenestrant/CDK4/6i (Ameera-1 trial); fulvestrant plus abemaciclib (Monarch 2 trial), and fulvestrant plus palbociclib (Paloma-3 trial)

TABLE 14

| | | Abema | | | | Fulvestrant/ | Camizes/ | Amcenes/ | Fulvestrant/ | |
|---|---|---|---|---|---|---|---|---|---|---|
| | LAS | Monarch | | | | Piqray | Palbo | CDKi | abeam | Fulv/Palbo |
| | Elaine 2¹ | 1² | Abema³ | | | Bylieve⁴ | Serena-1⁵ | Ameera-1⁶ | Monarch 2⁷ | Paloma-3⁸ |
| N | 29 | 132 | Total | Combo | Mono | 121 | 25 | 35 | 446 | 347 |
| | | | 87 | 70 | 17 | | | | | |
| CDK4/6I | Post | Naïve | Post | | | Post | Post | Naïve | Naïve | Naive |
| status | | | | | | | | | | |
| PFS (mos) | 13.9 | 6.0 | 5.3 | 5.1 | 5.4 | 7.3 | NA | 14.7 | 16.4 | 9.5 |
| (95% CI) | (7.4-NE) | (4.2-7.5) | (3.5-7.8) | (3.2-7.6) | (1.9-NR) | (5.6-8.3) | −4.3 mo.s* | (11-22.3) | | (9.2-11) |
| ORR | 50% | 19.7% | NR | NR | NR | 21% | 5.9% | 32.4% | 48.1% | 25% |
| CBR (≥24 | 69% | 41% | 36.8% | NR | NR | 45.5% | 28% | 74% | 72.2% | 67% |
| wks) | | | | | | | | | | |

¹Damodaran et al ASCO 2022;
²Dickler at al Clin Cancer Res 2017 Sep. 1: 23(17): 5219-5224;
³Wander et al, JNCCN, 10:6004, Mar. 24, 2021;
⁴Rugo et al Lancet Onc 2021;
⁵Oliveria ASCO 2022;
⁶Chandarlapaty ASCO 2021
⁷Sledge et al JCO 2017
⁸Cristofanilli et al Lancet Onc 2016
*Calculated from reported swimmer plot

### 6.2.16. Efficacy in Patients Whose Tumors Have Progressed on Prior CDK4/6i Therapy

A number of patients have been enrolled whose tumors had progressed on prior CDK4/6 inhibitor treatment. The combination of lasofoxifene and abemaciclib reduced progression of breast cancer in patients who had previously advanced on abemaciclib therapy.
Patient 29
   40 yr old with bone metastases: letrozole 3 yrs; letrozole/palbociclib 3 yrs; fulvestrant/abemaciclib 12 weeks; capecitabine 7 mos
   D538G 6.855% mutant allele fraction (MAF)
   Stable disease at 68 weeks
Patient 21
   42 yr old: Chemo/Herceptin; tamoxifen 10 yrs; letrozole/palbociclib 2 yrs 8 mos; abemaciclib 16 weeks
   24 mm liver mass
   Y537S 0.248% MAF
   Confirmed partial response at 48 weeks with liver lesion decreased 71% at 40 weeks
Patient 16
   78 yr old on letrozole/palbo for 2 yrs 2 mos; fulvestrant/abemaciclib 1 yr 3 mos; capecitabine 1 mos
   18 mm target liver lesion, pleural, LN and bone mets
   D538G 0.3% MAF
   Progressed at 40 weeks with stable disease (target lesion decreased 6%)
Patient 6
   59 yr old fulvestant/abemaciclib for 2 years; capecitabine 1 mos
   35 mm liver met
   D538G 1.28 MAF
   Progressed at 8 wks (liver lesion stable, but with new lesion)

### 6.3. Example 3: Second Interim Results of ELAINE 2 clinical trial

As of a second, later, interim date, two out of the four patients (50%) who had progressed on prior abemaciclib treatment continued to benefit from the combination of lasofoxifene and abemaciclib (laso/abema) treatment. Referring to FIG. 3, Patient 29 (2011-01), who had a D538G mutation, and prior treatments with palbociclib and fulvestrant, had continued response and stable disease to 88 weeks. Patient 21 (2005-02), who had a Y537S missense mutation, visceral metastases, and who had progressed on prior abemaciclib and palbociclib treatments, had stable disease in response to laso/abema until 56 weeks, at which time her cancer progressed. As shown in FIG. 3, patients who had had progression on other CDK 4/6 inhibitors (e.g., palbociclib, ribociclib) prior to enrollment also achieved clinical benefit (CB). Most had received palbociclib, and 70% (17/24) of such patients achieved CB, while 100% (2 of 2) who had been previously treated with ribociclib achieved CB. The data demonstrate that regardless of the prior CDK 4/6i, patients with an ESR1 mutation who had progressed on a CDK4/6i have a high probability of getting clinical benefit with combination treatment with lasofoxifene and abemaciclib. Patient 2004-03, who had visceral metastasis and had received prior palbociclib treatment, achieved CB and stable disease but withdrew early at 56 weeks for non-compliance (diamond). Table 15 summarizes second interim results of patients enrolled after previous abemaciclib progression.

TABLE 15
Second interim results of patients enrolled after previous abemaciclib progression

| | ESR1 Mut, | | | |
|---|---|---|---|---|
| Patient | MAF | Baseline disease | | |
| age | baseline/wk 4 | status | Prior mBC treatment | Current disease status |
| 40 y | D538G, | Bone metastases | LTZ/Palbo (3 yrs) | At 88 wks with SD |
| | 6.855%/ND | | Fulv/Abema (12 wks) | |
| | | | CAPE (7 mos) | |
| 42 y | Y537S, | 24 mm liver | LTZ/Palbo (2.7 yrs) | Stable disease until 56 |
| | 0.248%/ND | lesion | Abema (16 wks) | wks with confirmed PR |
| | | | | (liver lesion decreased |
| | | | | 71% at 40 wks) |
| 78 y | D538G, | 18 mm liver | LTZ/Palbo (2.2 yrs) | SD up to 40 wks (target |
| | 0.3%/ND | lesion, pleural, | Fulv/Abema (1.3 yrs) | lesion decreased 6%) |
| | | and bone | CAPE (1 mo) | |
| | | metastases | | |
| 59 y | D538G, | 35 mm liver | Fulv/Abema (2 yrs) | Progressed at 8 wks (liver |
| | 1.28%/1.926% | metastases | CAPE (1 mo) | lesion stable, but new |
| | | | | lesion noted) |

Patients who had had other relevant treatments prior to on-study laso/abema treatment also benefitted from the combination treatment. Referring to FIG. 3, Patient 2001-01, who had had prior alpelisib treatment, achieved CB with stable disease to 32 weeks. Patient 2009-02, who had had prior alpelisib, achieved CB with stable disease on the combination of lasofoxifene and abemaciclib to 36 weeks. Patient 2016-01, who had had prior PARP inhibitor (talzenna) treatment achieved CB and had confirmed partial response to 32 weeks on the combination of lasofoxifene and abemaciclib. Patient 2018-01, who had had prior ribociclib, achieved CB with stable disease at 60 weeks on combination treatment with lasofoxifene and abemaciclib. Patient 2004-06, who had previously been treated with ribociclib/fulvestrant for 9 months, showed confirmed partial response on lasofoxifene and abemaciclib, which was ongoing at 64 weeks.

Patients in this study are routinely achieving greater response on the combination of lasofoxifene and abemaciclib than on their preceding 2L and 3L therapies, which is surprising since, historically, subsequent lines of treatment often provide shorter duration benefit than was provided by the patient's earlier lines of treatment. As shown in FIG. 4, patients with laso/abema had longer duration of stable disease than on their respective preceding 2L and 3L therapies. As of the second interim date, subjects treated with laso/abema had an average of about 8.7 months duration of stable disease. All 29 patients had had preceding 2L therapies, which provided an average of about 4.2 months duration of stable disease. Nine of these subjects had also received 3L therapies prior to enrollment, providing an average of about 7.2 months of stable disease. Patients treated with the combination of lasofoxifene and abemaciclib have had, on average, an increase in duration of stable disease of about 51.2% as compared to their preceding 2L therapies, and an increase in duration of stable disease of about 16.9% as compared to their preceding 3L therapies. For example, Patient 2011-00001 (Patient 29) had stable disease to 22 months with laso/abema, as compared to 7 months of stable disease on preceding 2L therapy and 3 months of stable disease on preceding 3L therapy. Patient 2005-00002 (Patient 21) had stable disease to 14 months on lasofoxifene and abemaciclib, as compared to 4 months of stable disease on preceding 3L therapies. Patient 2015-00001 had stable disease to 19 months with laso/abema as compared to 10 months and 2 months of stable disease on preceding 2L and 3L therapies, respectively.

The results demonstrate acceptable tolerability, safety, and efficacy with combination of lasofoxifene and abemaciclib treatment in metastatic breast cancer patients harboring at least one ESR1 mutation who had progressed on one or more CDK4/6 inhibitors and endocrine therapies.

### 6.4. Example 4: ESR1 Mutations in Circulating Tumor DNA (ctDNA) From ELAINE 2 Clinical Trial Patients

This example investigates ESR1 mutations in circulating tumor DNA (ctDNA) in patients with ER⁺/HER2⁻ metastatic breast cancer (mBC) treated with lasofoxifene plus abemaciclib in the ELAINE 2 clinical trial. Data in this example demonstrate correlations of ESR1 mutant allele frequency (MAF) changes with clinical benefit (CB).

Use of long-term endocrine therapy (ET) for ER+ breast cancer often leads to acquired ESR1 mutations (mutESR1), causing endocrine resistance, tumor progression, and poor prognosis. An unmet clinical need exists for treating ER+ mBC patients with mutESR1, particularly after progression on CDK4/6 inhibitors (CDK4/6i). ELAINE 2 is an open-label, phase 2, multicenter trial evaluating safety and efficacy of lasofoxifene (LAS [selective estrogen receptor modulator]) plus abemaciclib (Abema [CDK4/6i], provided by Eli Lilly) in patients with ER+/HER2− and mutESR1 mBC who progressed after prior ET. Preliminary data with LAS plus Abema showed median progression-free survival of 55.7 weeks, objective response rate of 50%, and 24-week clinical benefit (CB) rate of 69%, with an acceptable safety and tolerability profile.

ELAINE 2 clinical trial patients with detectable ctDNA mutESR1 at baseline (BL) were analyzed. Oral LAS 5 mg/day and Abema 150 mg BID were taken until disease progression, death, unacceptable toxicity, or withdrawal from the study. ctDNA was assessed by the Sysmex-Inostics SafeSeq assay, which detects mutESR1 at low allele fractions at BL, every 4 weeks and end of treatment. MAF changes from BL to week 4 were characterized as decreased (decrease in ESR1 MAF or none detected [ND]), increased (increase in MAF), or equivocal (in polyclonal patients [>1 mutESR1] with some MAF increasing and decreasing trends). Correlations of MAF change at 4 weeks and with CB at 24 weeks were explored.

A total of 29 patients (median of 2 prior metastatic therapies: 97% CDK4/6i, 79% fulvestrant, 48% chemotherapy) had BL mutESR1 of Y537S (66%), D538G (45%), Y537N (28%), Y537C (10%), and other less frequently detected mutations; 14 (48.3%) patients were polyclonal. Twenty-six (26) of 29 patients had evaluable BL and week-4 ctDNA results: 21 patients had decreased MAF (81%, clearance in 14 [54% with ND]), 3 (12%) had increased, and 2 (8%) had equivocal ESR1 MAF changes as summarized in the Table 16 below.

TABLE 16
Change from baseline to week 4 in ESRI
MAF and clinical benefit at 24 weeks

| Clinical benefit at 24 | MAF change at 4 weeks (n = 26) | | |
|---|---|---|---|
| weeks | Decreased/ND | Increased | Equivocal |
| Yes | 17 (65%) | 2 (8%) | 1 (4%) |
| No | 4 (15%) | 1 (4%) | 1 (4%) |

| | |
|---|---|
| | Decreased/ND/Increased (n = 24) |
| Sensitivity* | 89.5% (95% CI, 65.5-98.2) |
| Specificity* | 20.0% (95% CI, 1.05-70.1) |
| PPV | 81.0% (95% CI, 57.4-93.7) |
| NPV | 33.3% (95% CI, 1.80-87.5) |
| | ND only (n = 14) |
| Sensitivity* | 86.7% (95% CI, 58.4-97.7) |
| Specificity* | 50.0% (95% CI, 9.50-90.5) |
| PPV | 92.9% (95% CI, 64.2-99.6) |
| NPV | 33.3% (95% CI, 1.80-87.5) |

CI, confidence interval; MAF, mutant allele fraction; ND, none detected; NPV, negative predictive value; PPV, positive predictive value.
*Sensitivity and specificity analyses do not include equivocal results.

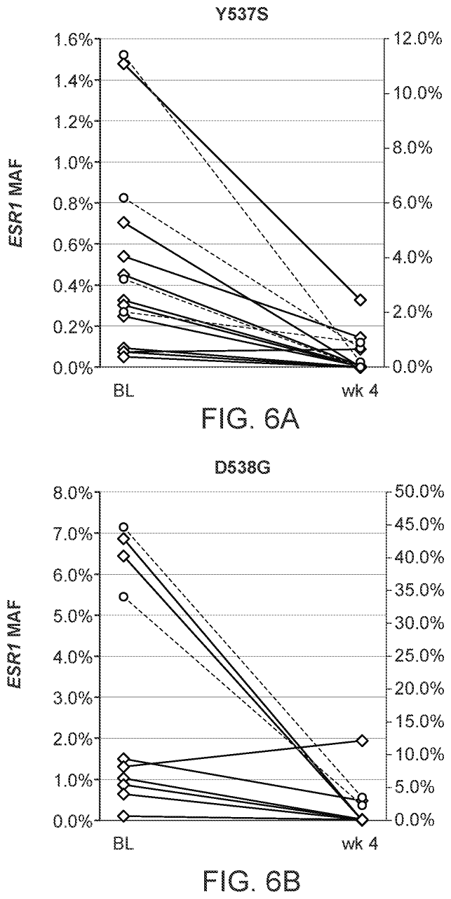

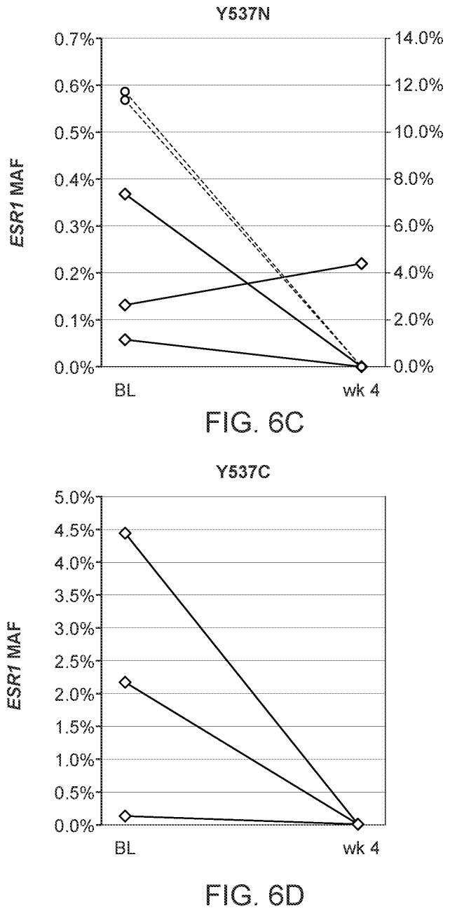

mESR1 clearance at week 4 was observed in 3 of the 4 patients who had previously progressed while taking prior Abema-based therapies with all 3 achieving CB. Decreased/cleared MAF was frequently observed for all the commonly detected mESR1 variants, including the Y537S, D538G, Y537N, and Y537C variants, after 4 weeks of LAS plus abema (FIG. 6). CB at 24 weeks was seen in 17 patients with decreased ESR1 MAF, 2 with an increase, and 1 with equivocal MAF change. A sensitivity of 89.5% and specificity of 20%, and a positive likelihood rPBatio (LR+) of 1.1 were calculated for predicting CB based on direction of ESR1 MAF change. The positive predictive value (PPV) for CB with decreased MAF was 81% and the negative predictive value (NPV) for an increased MAF was 33%. Of the 14 (54%) patients with ND ESR1 MAF, 13 had CB resulting in about 87% sensitivity, 50% specificity, 93% PPV, and 33% NPV with increased ESR MAF. mutESR1 clearance at week 4 had a similar sensitivity (about 87%) for CB prediction and a higher PPV (about 93%) compared with decreased MAF, with a LR+of 1.7. All 9 patients with an objective response (OR) showed complete mESR1 clearance (n=5) or 50%-93% decreases in ESR1 MAF (n=4) at week 4.

In the ELAINE 2 clinical trial, 81% of patients had decrease/cleared (ND) mutESR1 after four (4) weeks of LAS plus Abema, which correlated with clinical benefit. All mutESR1 detected appear targeted with this therapy. High sensitivity and favorable PPV were observed in patients with decreased MAF, and even more so in those with ND MAF; however, increased MAF was less specific and not as predictive of treatment failure.

In summary, analyses of ctDNA data in ELAINE 2 demonstrated that mutESR1 variants, including the difficult-to-treat Y537S, were decreased/cleared in most (about 81%) patients after 4 weeks of LAS plus Abema. Decreased/cleared ESR1 MAF was associated with CB and OR, with a high sensitivity (89%) and favorable PPV (81%) for predicting CB.

PPV was higher with mutESR1 clearance (93%). An increase in MAF was less specific and not as predictive of treatment failure. The results indicate robust target engagement of LAS plus Abema with mutESR1. Overall, the results demonstrate that ESR1 liquid biopsy evaluation is an appropriate non-invasive surrogate marker for monitoring patients' treatment response or resistance to this novel LAS-Abema combination.

### 6.5. Example 5: Oncogenic Mutations in Circulating Tumor DNA (ctDNA) From ELAINE 2 Clinical Trial Patients

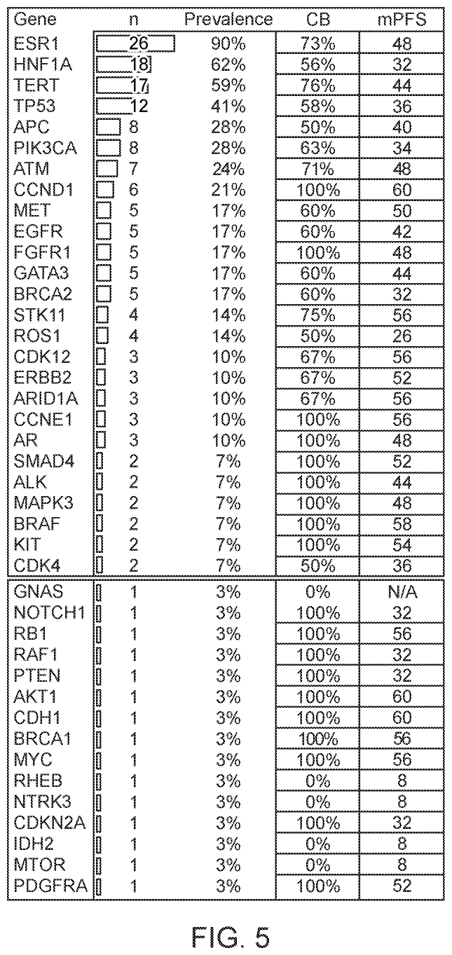

This example investigates oncogenic mutations of genes other than ESR1 in circulating tumor DNA (ctDNA) in patients with ER+/HER2− metastatic breast cancer (mBC) treated with lasofoxifene plus abemaciclib in the ELAINE 2 clinical trial. FIG. 5 summarizes the panel of genes being tested and that were present in the ELAINE 2 patient population. ESR1 gain of function mutation (mutESR1; top row) is included as a positive control. Data in this example demonstrate correlations of prevalence of oncogenic mutations of genes other than mutESR1 with clinical benefit (CB) and median progression-free survival (mPFS).

Approximately 5 mL whole blood samples were collected from individual ELAINE 2 clinical trial patient at baseline (BL) in Streck Cell-Free DNA Blood Collection Tubes (BCTs). The individual patient had previously been diagnosed by an oncologist as having ER+ mBC. To qualify for enrollment, the patient must have had either prior medical history indicating presence of mutESR1 or had been detected to have mutESR1, either intrinsic or acquired, using the assay as described in Example 4. Oral lasofoxifene 5 mg/day and abemaciclib 150 mg BID were taken until disease progression, death, unacceptable toxicity, or withdrawal from the study. Samples were processed for plasma isolation and cell free DNA (cfDNA) extraction, which may contain circulating tumor DNA. About 5-30 ng of cfDNA was used to prepare sequencing libraries which were enriched by hybridization capture. The enriched libraries were then sequenced using next generation sequencing, for example on the Illumina NextSeq 550 platform. Sequencing data were analyzed using a bioinformatics pipeline designed to detect single nucleotide variants (SNVs), insertions and deletions (indels), copy number amplifications (CNAs), and fusions. Both pathogenic (e.g., oncogenic) germline alteration and somatic alteration were detected.

As shown in FIG. 5, a total of 41 genes with one or more oncogenic mutations in the blood samples of the ELAINE 2 patient population were detected. Each gene mutation had a prevalence of at least about 3% of the patient population. The mutations were either germline, somatic or both. The most prevalent genes with one or more oncogenic mutations other than ESR1 were HNF1A (62%), TERT (59%), TP53 (41%), APC (28%), PIK3CA (28%), ATM (24%), CCND1 (21%), MET (17%), EFGR (17%), FGFR1 (17%), GATA3 (17%), and BRCA1 (17%). At least 43% of patients having oncogenic mutations in one or more of these genes achieved clinical benefit (CB)—stable disease and mPFS for at least 24 weeks—in response to the lasofoxifene and abemaciclib treatment. For example, about 100% of patients having an oncogenic mutation in CCND1 achieved CB and mPFS for at least 56 weeks, while 76% of patients having an oncogenic mutation in TERT achieved CB and mPFS for at least 44 weeks. In general, patients having an oncogenic mutation in at least one of the genes selected from HNF1A, TERT, GATA3, CDK12, MAPK3, TP53, PIK3CA, APC, ATM, MET, CCND1, EGFR, ROS1, STK11, FGFR1, ARID1A, CCNE1, AR, ALK, ERBB2, KIT, CDK4, SMAD4, NOTCH1, RB1, BRAF, RAF1, PTEN, AKT1, CDH1, BRCA1, MYC, CDKN2A, or PDGFRA achieved mPFS for at least 24 weeks. Patients having one or more mutations in at least one of the genes selected from CCND1, FGFR1, CCNE1, AR, ALK, MAPK3, KIT, SMAD4, NOTCH1, RB1, BRAF, RAF1, PTEN, AKT1, CDH1, BRCA1, MYC, CDKN2A, or PDGFRA achieved 100% clinical benefit and mPFS for at least 32 weeks. It is noted that patients having oncogenic mutations in one or more of the genes selected from GNAS, RHEB, NTRK3, IDH2, and/or mTOR had no CB and had mPFS for 8 weeks or less.

Interestingly, in these baseline measurements, an ESR1 mutation was detected in only about 90% of the enrolled subjects. In at least some of the subjects whose enrollment in the clinical trial was based on prior medical history of ESR1 mutation rather than detection of ESR1mut at enrollment, this likely can be attributed to the patient's response to prior treatments. This would be consistent with our observations in the ELAINE 2 clinical trial itself, where approximately 68.9% (20 out of 29) of subjects displayed decreased or clear (ND) ESR1 mutation at week 4 on LAS+a bema treatment.

For example, Patient 29 (2011-00001) had an ESR1 D538G mutation at baseline, following prior (pre-enrollment) abemaciclib treatment and other first line or second line endocrine therapies (FIGS. 1 and 3). Patient 29 became ND for ESR1 mutation in ctDNA at week 4 on lasofoxifene and abemaciclib combination treatment. Patient 29 continues to respond to the treatment and is having clinical benefit with stable disease for at least 88 weeks (FIG. 3).

Patient 16 (2003-00001) had prior medical history indicating presence of the ESR1 gain of function mutation D538G. Patient 16 also had received prior abemaciclib treatment and other first line or second line endocrine therapies (FIGS. 1 and 3). At baseline, Patient 16 had no detectable mutESR1 (ND for ESR1 mutation) using the assay described in Example 4. At baseline, Patient 16 was detected to have oncogenic mutations in TERT, ATM, and MAPK3 (data not shown). Patient 16 maintained ND for ESR1 mutation in ctDNA until end of treatment at week 40.

The data in this example demonstrate that lasofoxifene in combination with abemaciclib is effective in treating metastatic ER+ breast cancers having an ESR1 gene mutation and an oncogenic mutation in one or more genes other than ESR1; and that lasofoxifene in combination with abemaciclib is effective in treating metastatic ER+ breast cancers that do not have a detectable ESR1 mutation and have one or more oncogenic mutations in a gene other than ESR1.

### 6.6. Example 6: Third Interim Results of ELAINE 2 Clinical Trial

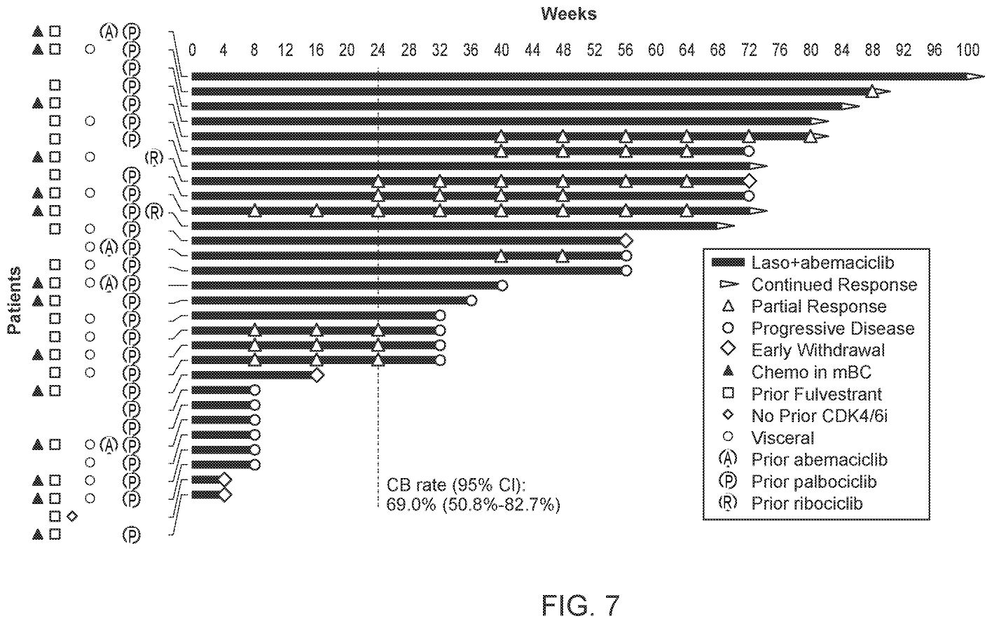

As of a third, later, interim date, 20 out of 29 patients (about 69%) had CB from treatment with the combination of lasofoxifene and abemaciclib (laso/abema). Referring to FIG. 7, which is an updated Swimmer Plot, 11 out of 20 patients (55%) who had CB had updated status as compared to FIG. 3. Among of these patients, 8 out of 11 patients (about 72.7%) had continued or partial response and stable disease at week 68 to 100 since the last update. For example, Patient 29 (2011-01) continued to have continued response and stable disease to 100 weeks, while Patient 2005-01 had continued response and stable disease to 84 weeks, Patient 2002-01 had continued response and stable disease to 80 weeks, Patients 2001-05 and 2016-03 had continued response and stable disease to 72 weeks, Patient 2018-01 had continued response and stable disease to 68 weeks, and Patient 2017-01 had partial response and stable disease to 80 weeks. Patient 2015-01 had partial response and stable disease to 88 weeks. Two (2) out of 20 patients (10%) had progressed at week 72 (Patient 2001-02 and Patient 2014-01), and one patient (5%) had an early withdrawal (Patient 2004-06). As shown in Table 17 below, 7 out of 11 patients (about 63.6%) had the difficult-to-treat Y537S mutation at baseline. The data show that all 7 patients had continued or partial response to the combination of laso/abema treatment, and had stable disease up to at least 72 weeks. Laso/abema efficacy is observed even in patients who had visceral metastases at baseline (about 42.8%, 3 out of 7). At least 7 out of 11 patients (about 63.6%) had oncogenic mutations in one or more genes other than ESRD, and at least one of the oncogenic mutations had a prevalence of over 20% in the patient population (e.g., HNF1A, TERT, TP53, APC, PIK3CA, ATM, CCND1, see also FIG. 5).

TABLE 17
Efficacy of combination of laso/abema treatment on patients
with ESR1, other oncogenic mutations and prior treatments

| | | Other | Continued | Partial | | | | |
|---|---|---|---|---|---|---|---|---|
| | | oncogenic | response/ | response/ | | Early | Prior | |
| | ESR1 | mutations | stable | stable | Progressed | withdrawal | CDK4/6i | Visceral |
| Patient | (ctDNA) | (ctDNA) | (week) | (week) | (week) | (week) | treatment | metastases |
| 2011-01 | D538G | TBD | 100 | n/a | n/a | n/a | palbociclib, | No |
| | | | | | | | abemaciclib | |
| 2015-01 | Y537S | TBD | | 88 | n/a | n/a | palbociclib | Yes |
| 2005-01 | Y537S, | HNF1A, | 84 | n/a | n/a | n/a | palbociclib | No |
| | Y537N, | TERT, | | | | | | |
| | D538G | TP53, | | | | | | |
| | | ATM, | | | | | | |
| | | CCND1, | | | | | | |
| | | MET, | | | | | | |
| | | EGFR1, | | | | | | |
| | | GATA3, | | | | | | |
| | | CCNE1, | | | | | | |
| | | CDK4 | | | | | | |
| 2002-01 | Y537S, | HNF1A, | 80 | n/a | n/a | n/a | palbociclib | No |
| | Y537N, | AKT1, | | | | | | |
| | D538G | CDH1 | | | | | | |
| 2017-01 | E380Q, | TERT, | n/a | 80 | n/a | n/a | palbociclib | No |
| | L469V | APC, | | | | | | |
| | | CCNE1, | | | | | | |
| | | ALK | | | | | | |
| 2001-02 | Y537S, | PIK3CA, | n/a | n/a | 72 | n/a | palbociclib | Yes |
| | Y537N, | CDK12, | | | | | | |
| | Y537D, | BRCA1 | | | | | | |
| | D538G | | | | | | | |
| 2001-05 | Y537S | APC, | 72 | n/a | n/a | n/a | palbociclib | No |
| | | PIK3CA, | | | | | | |
| | | BRCA2, | | | | | | |
| | | AR | | | | | | |
| 2004-06 | Y537S | HNF1A, | n/a | n/a | n/a | 72 | palbociclib | Yes |
| | | TERT, | | | | | | |
| | | GATA3, | | | | | | |
| | | ROS1, | | | | | | |
| | | KIT | | | | | | |
| 2014-01 | Y537S, | TP53, | n/a | n/a | 72 | n/a | palbociclib | No |
| | Y537N, | CCND1, | | | | | | |
| | D538G | MET, | | | | | | |
| | | ARID1A, | | | | | | |
| | | BRAF | | | | | | |
| 2016-03 | D538G | TBD | n/a | 72 | n/a | n/a | palbociclib | Yes |
| 2018-01 | Y537C | TBD | 68 | n/a | n/a | n/a | palbociclib, | No |
| | | | | | | | ribociclib | |

*TBD: oncogenic mutations to be determined

Referring to Table 17, it is noted that patients who have mutations that have previously been associated with endocrine resistance or CDK4/6i resistance achieved clinical benefit (CB) with stable disease and showed complete or partial response for at least 72 weeks. For example, patients who have at least one of FGFR1, ERBB2, CCND1, CCNE1, ARD1A, PIK3CA and TP53, had consistent robust clinical response to the lasofoxifene/abemaciclib combination in Elaine 2. Patient 2005-01, who had TP53, CCND1 and CCNE1 mutations or CNV, had complete response and stable disease to 84 weeks. Patient 2014-01, who had TP53, CCND1 and ARID IA mutations or CNV, had partial response and progressed at 72 weeks. Patient 2001-02, who had PIK3CA mutation or CNV, had partial response and progressed at 72 weeks. Patient 2001-05, who had PIK3CA mutation or CNV, had complete response and stable disease to 72 weeks. It is noted that these four patients also have the difficult-to-treat Y537S mutESR1 variant.

These results are unexpected and demonstrate that combination treatment with lasofoxifene and abemaciclib is efficient in reducing or preventing tumor progression in patients who have higher risk of developing resistance to endocrine therapy or CDK4/6i treatment, or who have developed resistance to endocrine therapy or CDK4/6i treatment.

The results are consistent with our observations presented in Example 3 and further demonstrate acceptable tolerability, safety, and efficacy with combination treatment with lasofoxifene and abemaciclib in metastatic breast cancer patients harboring at least one ESR1 mutation and often at least one or more mutations in other genes, who had progressed on one or more CDK4/6 inhibitors, and/or one or more endocrine therapies.

### 6.7. Example 7: Copy Number Variations of ESR1 Mutations and Oncogenic Mutations in Circulating Tumor DNA (ctDNA) from ELAINE 2 Clinical Trial Patients

This example investigates copy number variations (CNVs) of ESR1 mutations and oncogenic mutations in circulating tumor DNA (ctDNA) in patients with ER+/HER2− metastatic breast cancer (mBC) treated with lasofoxifene plus abemaciclib in the ELAINE 2 clinical trial as described in Example 5. Data in this example demonstrate correlations of presence of ESR1 mutation variants and/or oncogenic mutation variants with clinical benefit (CB).

A total of 29 samples received from 29 patients (Batch 1:25 samples/patients; Batch 2: 4 samples/patients) were collected from the ELAINE 2 clinical trial. Patients were administered oral lasofoxifene 5 mg/day and abemaciclib 150 mg BID until disease progression, death, unacceptable toxicity, or withdrawal from the study. Samples were processed for plasma isolation and cell free DNA (ctDNA) extraction and sequencing as described in Example 5. Sequencing data were analyzed using a bioinformatics pipeline designed to detect single nucleotide variants (SNVs), insertions and deletions (indels), copy number variations (CNVs), and fusions. In Batch 1, copy number amplifications (CNAs) were detected in 19 genes, including CCND1. Amplification type was annotated as focal, aneuploidy, or amplification where focal/aneuploidy status is indeterminate. The results are provided in Table 18.

TABLE 18
Copy number variations of ESRI mutations and oncogenic mutations

| | Batch 1 | | | Batch 2 | | |
|---|---|---|---|---|---|---|
| | | Number of | | | Number of | |
| | | samples | | | samples | |
| Mutation | Alteration | (n = 25) | | Alteration | (n = 4) | |
| ESR1 | L536P, L536H, L536R, | 22 | (88%) | Y537S, Y537C, | 4 | (100%) |
| | Y537S, Y537N, Y537C, | | | D538G | | |
| | D538G, E380Q | | | | | |
| PIK3CA | E542K, E545K, H1047R, | 8 | (32%) | n/a | n/a | |
| | M1043I, Q546K, G118D | | | | | |
| ERBB2 | S310F | 1 | (4%) | M1014R | 1 | (4%) |
| CCND1 | Focal amplification | 5 | (20%) | Focal amplification | 1 | (25%) |

| | | | | | |
|---|---|---|---|---|---|
| FGFR1 | n/a | n/a | Amplification | 2 | (50%) |

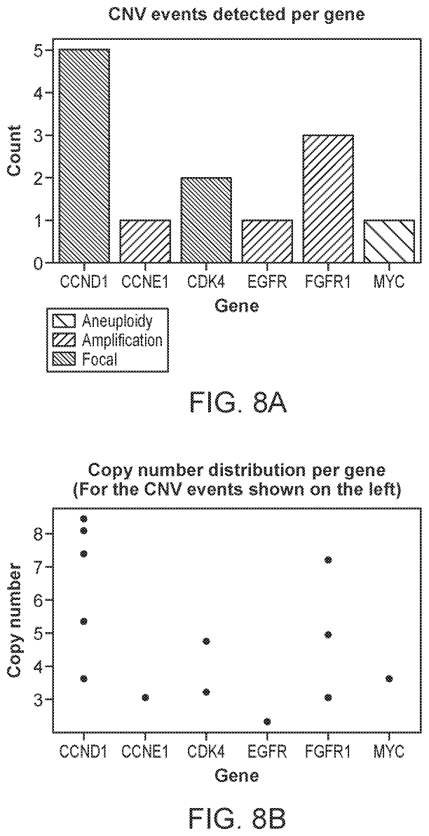

FIG. 8 illustrates an example of copy number variation events detected per gene. FIG. 8 left panel shows copy number variation (CNV) events detected per gene, including CCND1, CCNE1, CDK4, EGFR, FGFR1, MYC. FIG. 8 right panel shows copy number distribution per gene for the CNV events shown on the left panel. Five CNV events and over eight copy numbers were detected in CCND1, three CNV events and over seven copy numbers were detected in FGFR1, while two CNV events and about five copy numbers were detected in CDK4.

### 6.8. Example 8: Interim Results of Oncogenic Mutations in Circulating Tumor DNA (ctDNA) from ELAINE 2 Clinical Trial Patients

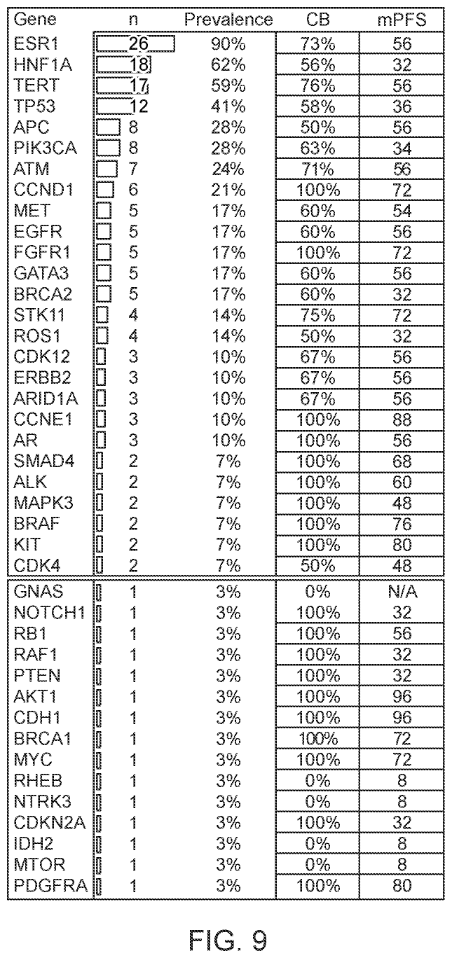

This example provides, as of an interim date later than the date of Example 5, oncogenic mutations of genes other than ESR1 present in circulating tumor DNA (ctDNA) in patients with ER+/HER2− metastatic breast cancer (mBC) treated with lasofoxifene plus abemaciclib in the ELAINE 2 clinical trial as described in Example 5. The results are summarized in FIG. 9. ESR1 gain of function mutation (mutESR1; top row) is included for completeness.

As shown in FIG. 9, a total of 41 genes with one or more oncogenic mutations in the blood samples of the ELAINE 2 patient population were detected. At least 87.8% (36 out of 41) of patients having oncogenic mutations in one or more of these genes achieved clinical benefit (CB)—defined as stable disease ≥24 weeks, or confirmed partial or complete response—in response to the lasofoxifene and abemaciclib treatment. Patients having one or more mutations in TERT, APC, ATM, CCND1, MET, EGFR, FGFR1, GATA3, STK11, ROS1, ERBB2, CCNE1, AR, SMAD4, ALK BRAF, KIT, CDK4, AKT1, CDH1, BRCA1, MYC, and PDGFRA continued to achieve CB and mPFS. For example, about 100% of patients having an oncogenic mutation in CCND1 continued to achieve CB and mPFS for at least 72 weeks, 100% of patients having an oncogenic mutation in FGFR1 continued to achieve CB and mPFS for at least 72 weeks, while 76% of patients having an oncogenic mutation in TERT continued to achieve CB and mPFS for at least 56 weeks. Patients having one or more mutations in TP53 or PIK3CA achieved CB and mPFS for at least 36 weeks and 34 weeks, respectively. Notably, patients having co-existing copy number variant (CNV) in CCND1 and FGFR1 and alterations in PIK3CA and TP53 responded to lasofoxifene and abemaciclib.

The data in this example demonstrate that lasofoxifene in combination with abemaciclib is effective in treating metastatic ER+ breast cancers having an ESR1 gene mutation and an oncogenic mutation in one or more genes other than ESR1, and in maintaining CB and mPFS in this subset of patients, and in patients who have metastatic ER+ breast cancers that do not have a detectable ESR1 mutation and have one or more oncogenic mutations in a gene other than ESR1.

### 6.9. Example 9: Fourth Interim Results of ELAINE 2 Clinical Trial

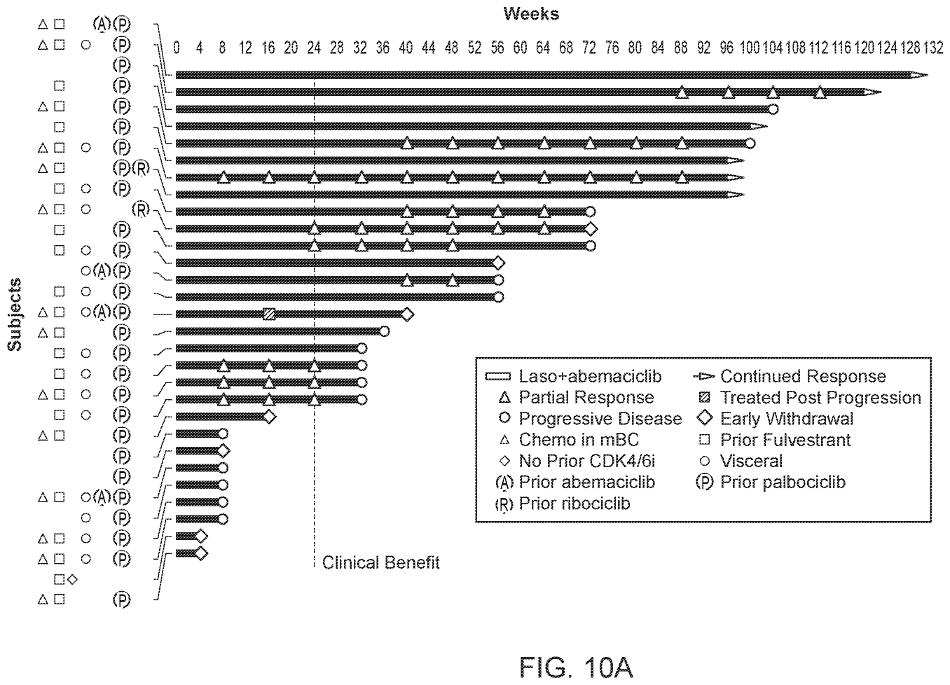

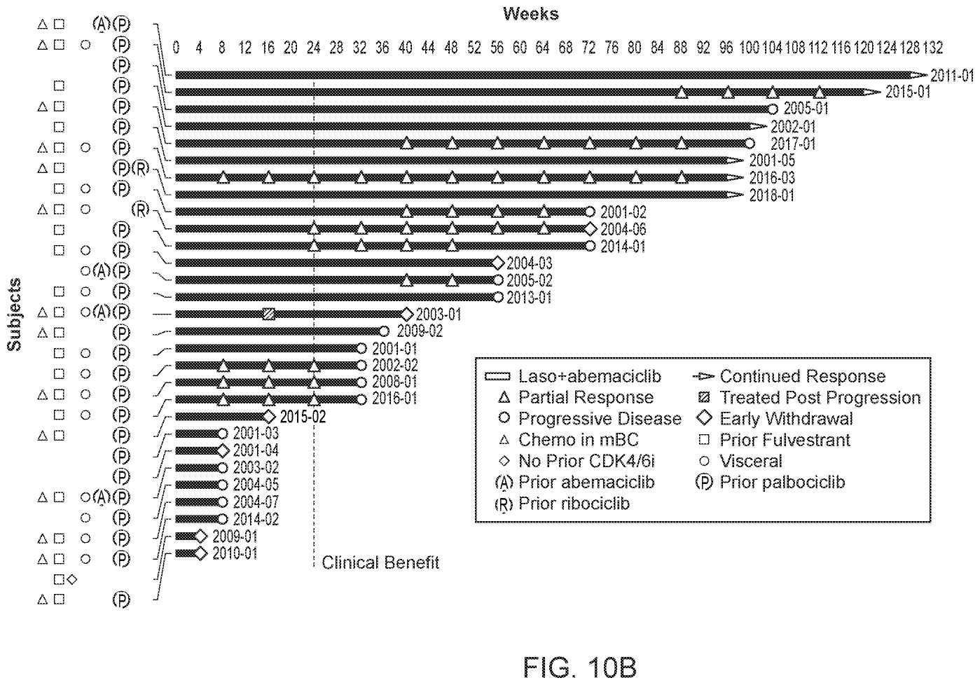

As of a fourth, later date than the date of Example 6, 20 out of 29 patients (about 68.9%) continued to have CB from treatment with the combination of lasofoxifene and abemaciclib (laso/abema). Referring to FIGS. 10A-10B, which is an updated Swimmer Plot, 8 out of 20 patients (40%) who had CB had updated status as compared to FIG. 7. Among of these patients, 6 out of 8 patients (75%) had continued or partial response and stable disease at week 96 to 128 since the last update, while 2 out of 8 patients (25%) had continued or partial response and progressive disease. For example, Patient 29 (2011-01) continued to have continued response and stable disease to 128 weeks, while Patient 2002-01 had continued response and stable disease to 100 weeks, Patients 2001-05 and 2018-01 had continued response and stable disease to 96 weeks. Patient 2015-01 had partial response and stable disease to 120 weeks. Patient 2016-03 had partial response and stable disease to 96 weeks. Patient 2005-01 had continued response but progressed at 104 weeks. Patient 2017-01 had partial response and progressed at 100 weeks. Table 19 summarizes efficacy of lasofoxifene/abemaciclib combination in patients who achieved clinical benefit, including patients described in Table 17. As shown, 13 out of 20 patients (65%) had the difficult-to-treat Y537S mutation at baseline but had continued or partial response and stable disease up to 120 weeks (e.g., Patient 2015-01). Lasofoxifene/abemaciclib efficacy is observed even in patients who had visceral metastases at baseline (60%, 12 out of 20). All 20 patients (100%) who had clinical benefit also had oncogenic mutations in one or more genes other than ESRD, and at least one of the oncogenic mutations had a prevalence of over 17% in the patient population (e.g., HNF1A, TERT, TP53, APC, PIK3CA, ATM, CCND1, FGFR1, see also FIG. 9).

TABLE 19
Efficacy of combination of laso/abema treatment on patients
with ESR1, other oncogenic mutations and prior treatments

| | | Other | Continued | Partial | | | | |
|---|---|---|---|---|---|---|---|---|
| | | oncogenic | response/ | response/ | | Early | Prior | |
| | ESR1 | mutations | stable | stable | Progressed | withdrawal | CDK4/6i | Visceral |
| Patient | (ctDNA) | (ctDNA) | (week) | (week) | (week) | (week) | treatment | metastases |
| 2011-01 | D538G | CCND1, | 128 | n/a | n/a | n/a | palbociclib, | No |
| | | TP53, | | | | | abemaciclib | |
| | | TERT, | | | | | | |
| | | HNF1A | | | | | | |
| 2015-01 | Y537S | FGFR1, | n/a | 120 | n/a | n/a | palbociclib | Yes |
| | | TP53 | | | | | | |
| 2005-01 | Y537S, | HNF1A, | n/a | n/a | 104 | n/a | palbociclib | No |
| | Y537N, | TERT, | | | | | | |
| | D538G | TP53, | | | | | | |
| | | ATM, | | | | | | |
| | | CCND1, | | | | | | |
| | | MET, | | | | | | |
| | | EGFR1, | | | | | | |
| | | GATA3, | | | | | | |
| | | CCNE1, | | | | | | |
| | | CDK4, | | | | | | |
| | | KIT | | | | | | |
| 2002-01 | Y537S, | HNF1A, | 100 | n/a | n/a | n/a | palbociclib | No |
| | Y537N, | AKT1, | | | | | | |
| | D538G | CDH1 | | | | | | |
| 2017-01 | E380Q, | TERT, | n/a | n/a | 100 | n/a | palbociclib | No |
| | L469V | APC, | | | | | | |
| | | CCNE1, | | | | | | |
| | | ALK | | | | | | |
| 2001-02 | Y537S, | PIK3CA, | n/a | n/a | 72 | n/a | palbociclib | Yes |
| | Y537N, | CDK12, | | | | | | |
| | Y537D, | BRCA1, | | | | | | |
| | D538G | FGFR1, | | | | | | |
| | | CCND1 | | | | | | |
| 2001-05 | Y537S | APC, | 96 | n/a | n/a | n/a | palbociclib | No |
| | | PIK3CA, | | | | | | |
| | | BRCA2, | | | | | | |
| | | AR | | | | | | |
| 2004-06 | Y537S | HNF1A, | n/a | n/a | n/a | 72 | palbociclib | Yes |
| | | TERT, | | | | | | |
| | | GATA3, | | | | | | |
| | | ROS1, | | | | | | |
| | | KIT | | | | | | |
| 2014-01 | Y537S, | TP53, | n/a | n/a | 72 | n/a | palbociclib | No |
| | Y537N, | CCND1, | | | | | | |
| | D538G | MET, | | | | | | |
| | | ARID1A, | | | | | | |
| | | BRAF | | | | | | |
| 2016-03 | D538G | TERT, | n/a | 96 | n/a | n/a | palbociclib | Yes |
| | | BRAF, | | | | | | |
| | | ATM, | | | | | | |
| | | EGFFR, | | | | | | |
| | | BRCA2, | | | | | | |
| | | ERBB2, | | | | | | |
| | | STK11, | | | | | | |
| | | PDGFRA, | | | | | | |
| | | APC, TP53 | | | | | | |
| 2018-01 | Y537C | FGFR1, | 96 | n/a | n/a | n/a | palbociclib, | No |
| | | TERT, | | | | | ribociclib | |
| | | ATM, | | | | | | |
| | | HNF1A, | | | | | | |
| | | SMAD4 | | | | | | |
| 2001-01 | Y537S, | PIK3CA, | n/a | n/a | 32 | n/a | palbociclib | Yes |
| | D538G | RAF1, | | | | | | |
| | | PTEN, | | | | | | |
| | | APC | | | | | | |
| 2002-02 | Y537N, | TERT, | n/a | n/a | 32 | n/a | palbociclib | Yes |
| | L536H, | CCNE1, | | | | | | |
| | D538G, | EGFR | | | | | | |
| | E380Q | | | | | | | |
| 2004-03 | Y537S | CDK12, | n/a | n/a | n/a | 56 | palbociclib | Yes |
| | | TERT, | | | | | | |
| | | ROS1, | | | | | | |
| | | ERBB2, | | | | | | |
| | | HNF1A, | | | | | | |
| | | MAPK3, | | | | | | |
| | | RB1 | | | | | | |
| 2005-02 | Y537S | TERT, | n/a | n/a | 56 | n/a | abemaciclib, | Yes |
| | | STK11, | | | | | palbociclib | |
| | | PIK3CA, | | | | | | |
| | | AR, | | | | | | |
| | | ARID1A | | | | | | |
| 2008-01 | Y537S, | HNF1A, | n/a | n/a | 32 | n/a | palbociclib | Yes |
| | D538G | BRCA2, | | | | | | |
| | | CDKN2A, | | | | | | |
| | | AR, | | | | | | |
| | | CCND1, | | | | | | |
| | | FGFR1 | | | | | | |
| 2009-02 | Y537S | PIK3CA, | n/a | n/a | 36 | n/a | palbociclib | No |
| | | TP53, | | | | | | |
| | | TERT, | | | | | | |
| | | MET | | | | | | |
| 2013-01 | D538G | ATM, | n/a | n/a | 56 | n/a | palbociclib | Yes |
| | | SMAD4 | | | | | | |
| 2016-01 | Y537S, | TERT, | n/a | n/a | 32 | n/a | palbociclib | Yes |
| | Y537C, | TP53, | | | | | | |
| | L536P, | HNF1A, | | | | | | |
| | D538G | NOTCH1, | | | | | | |
| | | ALK, | | | | | | |
| | | CCND1, | | | | | | |
| | | FGFR1 | | | | | | |
| 2003-01 | D538G | TERT, | n/a | n/a | Progressed | n/a | abemaciclib, | Yes |
| | | MAPK3, | | | at 14 weeks | | palbociclib | |
| | | ATM | | | and | | | |
| | | | | | remained | | | |
| | | | | | on study | | | |
| | | | | | with stable | | | |
| | | | | | disease | | | |
| | | | | | until 40 | | | |
| | | | | | weeks | | | |

Referring to Table 19, it is noted that patients who have mutations that have previously been associated with endocrine resistance or CDK4/6i resistance, such as FGFR1, ERBB2, CCND1, CCNE1, ARD1A, PIK3CA and TP53, continued to have consistent robust clinical response to the lasofoxifene/abemaciclib combination in Elaine 2. Subject 2011-01, who has D538G mutESR1 variant and TP53 and CCND1 mutations or copy number variant (CNV), continued to have complete response and stable disease to 128 weeks. Patient 2017-01, who has E380Q and L469V mutESR1 variants and CCNE1 mutation or CNV, continued to have partial response and progressed at 100 weeks. Patient 2016-03, who has D538G mutESR1 variant and ERBB2 mutation or CNV, continued to have partial response and stable disease at 96 weeks. Patient 2002-02, despite having multiple mutESR1 variants, including Y537N, L536H, D538G, and E380Q, and who had CCNE mutation or CNV, had partial response until progression at 32 weeks.

It is noted that patients who have the difficult-to-treat Y537S mutESR1 variant continued to have consistent robust clinical efficacy to the lasofoxifene/abemaciclib combination. Patient 2015-01, who has Y537S mutESR1 variant and FGFR1 and TP53 mutations or CNV, continued to have partial response and stable disease to 120 weeks. Patient 2005-01, who has Y537S, Y537N, and D538G mutESR1 variants and TP53, CCND1 and CCNE1 mutations or CNV, continued to have complete response and stable disease and progressed at 104 weeks. Patient 2001-02, who has Y537S, Y537N, Y537D, and D538G mutESR1 variants and PIK3CA, FGFR1 and CCND1 mutations or CNV, had partial response and progressed at 72 weeks. Patient 2014-01, who has Y537S, Y537N, and D538G mutESR1 variants and TP53, CCND1 and ARID1A mutations or CNV, had partial response and progressed at 72 weeks. Patient 2004-03, who has Y537S mutESR1 variant and ERBB2 mutation or CNV, had complete response and stable disease at 56 weeks, but had early withdrawal due to non-compliance. Patient 2005-02, who has a Y537S mutESR1 variant and PIK3CA and ARID1A mutations or CNV, had partial response and progressed at 56 weeks. Patient 2008-01, who has Y537S and D538G mutESR1 variants and CCND1 and FGFR1 mutations or CNV, had partial response and progressed at 32 weeks. Patient 2016-01, who has Y537S, Y537C, L536P and D538G mutESR1 variants and TP53, CCND1 and FGFR1 mutations or CNV, had partial response and progressed at 32 weeks. Patient 2001-05, who has Y537S mutESR1 variant and PIK3CA mutation or CNV, continued to have complete response and stable disease to 96 weeks. Patient 2001-01, who has Y537S and D538G mutESR1 variants and PIK3CA mutation or CNV, had partial response and progressed at 32 weeks. Patient 2009-02, who has Y537S mutESR1 variant and TP53 and PIK3CA mutations or CNV, had complete response and stable disease and progressed at 36 weeks.

Patients who had target lesion at baseline were monitored for change on study. It is noted that 10 out of 20 (50%) had confirmed partial response and achieved clinical benefit. Patient 2001-01 had target lesion in the lung (11 mm) at baseline and the lesion decreased by 55% at 64 weeks. Patient 2002-02 had target lesions in the liver (20 mm), lungs (23 mm, 29 mm) at baseline (sum diameter of 72 mm) and the lesion decreased by 74% at 32 weeks. Patient 2004-06 had target lesions in liver, bone, and pleural cavity (sum diameter of 79 mm) at baseline and the lesion decreased by 47% at 64 weeks. Patient 2005-02 had target lesions in liver (sum diameter of 24 mm) at baseline and the lesion decreased by 33% at 56 weeks. Patient 2008-01 had target lesions in liver (sum diameter of 56 mm) at baseline and the lesion decreased by 50% at 32 weeks. Patient 2014-01 had target lesions in liver and spleen (41 mm) and had the lesions decreased by 22% at 64 weeks. Patient 2015-01 had target lesions in the liver (sum diameter of 56 mm) and the lesion decreased by 52% at 104 weeks. Patient 2016-01 had target lesions in the liver (47 mm) and the lesion decreased by 47% at 32 weeks. Patient 2016-03 had target lesion in the liver (sum diameter of 67 mm) and the lesion decreased by 67% at 88 weeks. Patient 2017-01 had target lesion in left paraceliac (sum diameter 15 mm) and the lesion decreased by 40% at 96 weeks. The data demonstrate that combination treatment with lasofoxifene/abemaciclib is effective in reducing tumor progression and/or inhibiting tumor growth.

The results are consistent with our observations in Examples 3 and 7 and further demonstrate acceptable tolerability, safety, and efficacy with combination treatment with lasofoxifene and abemaciclib in metastatic breast cancer patients harboring at least one ESR1 mutation and often at least one or more mutations in other genes, who had progressed on one or more CDK4/6 inhibitors, and/or one or more endocrine therapies. Additionally, the results further demonstrate that combination treatment with lasofoxifene and abemaciclib is effective in patients harboring the difficult-to-treat mutESR1 variants. The results are unexpected in patients who also have one or more oncogenic mutations or CNV of biomarkers associated with endocrine therapy or CDK4/6i resistance.

### 7. EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

1. A method of treating breast cancer in a patient, comprising:
administering to the patient an effective amount of lasofoxifene or a pharmaceutically acceptable salt thereof and an effective amount of a CDK4/6 inhibitor (CDK4/6i),
wherein the breast cancer:
(i) is estrogen receptor positive (ER⁺);
(ii) has at least one gain of function missense mutation within the ligand binding domain (LBD) of the Estrogen Receptor 1 (ESR1) gene; and
(iii) has progressed during prior CDK4/6 inhibitor therapy.

2. The method of claim 1, wherein the ER⁺ breast cancer is HER2⁻.

3. The method of claim 2, wherein the ER⁺ breast cancer is locally advanced.

4. The method of claim 2, wherein the ER⁺ breast cancer is metastatic.

5. The method of claim 1, wherein lasofoxifene is administered as lasofoxifene tartrate.

6. The method of claim 5, wherein lasofoxifene tartrate is administered orally at 5 mg lasofoxifene/day.

7. The method of claim 1, wherein the CDK4/6i administered to the patient is selected from palbociclib, ribociclib, and abemaciclib.

8. The method of claim 7, wherein the CDK4/6i administered to the patient is abemaciclib.

9. The method of claim 8, wherein abemaciclib is administered orally at 50 mg to 200 mg BID.

10. The method of claim 9, wherein abemaciclib is administered orally at 100 mg to 200 mg BID.

11. The method of claim 10, wherein abemaciclib is administered orally at 150 mg BID.

12. The method of claim 1, wherein the prior administered CDK4/6 inhibitor is selected from palbociclib, ribociclib, and abemaciclib.

13. The method of claim 12, wherein the prior administered CDK4/6 inhibitor is abemaciclib.

14. The method of claim 13, wherein the cancer has previously been determined to have at least one gain of function missense mutation within the ligand binding domain (LBD) of the Estrogen Receptor 1 (ESR1) gene.

15. The method of claim 1, further comprising the earlier step of:
determining that the patient has at least one gain of function missense mutation within the ligand binding domain (LBD) of the Estrogen Receptor 1 (ESR1) gene.

16. The method of claim 1, wherein the at least one of gain of function missense mutation is in any one of amino acids D538, Y537, L469, L536, P535, V534, 5463, V392, and E3 80.

17. The method of claim 16, wherein the at least one gain of function missense mutation is in any one of amino acids D538G, Y537S, Y537N, Y537C, Y537Q, Y537C, L469V, L536R, L536Q, P535H, V534E, S463P, V392I, and E380Q.

18. The method of claim 4, wherein the ER⁺ breast cancer is visceral metastatic.

19. The method of claim 1, wherein the breast cancer had progressed on one or more prior endocrine therapies.

20. The method of claim 19, wherein the prior endocrine therapy is a selective ER degrader (SERD), a selective ER modulator (SERM) other than lasofoxifene, an aromatase inhibitor (AI), an mTOR inhibitor, and/or a PI3K inhibitor.

21. The method of claim 20, wherein the SERD is fulvestrant.

22. The method of claim 1, wherein the patient has complete or partial response to the treatment with lasofoxifene or a pharmaceutically acceptable salt thereof and a CDK4/6i.

23. A method of treating breast cancer in a patient, comprising:
administering to the patient an effective amount of lasofoxifene or a pharmaceutically acceptable salt thereof and an effective amount of a CDK4/6 inhibitor (CDK4/6i),
wherein the breast cancer:
(i) is estrogen receptor positive (ER⁺);
(ii) has at least one gain of function missense mutation within the ligand binding domain (LBD) of the Estrogen Receptor 1 (ESR1) gene; and
(iii) has an oncogenic mutation in one or more genes other than the ESR1 gene.

24. A method of treating breast cancer in a patient, comprising:
administering to the patient an effective amount of lasofoxifene or a pharmaceutically acceptable salt thereof and an effective amount of a CDK4/6 inhibitor (CDK4/6i),
wherein the breast cancer:
(i) is estrogen receptor positive (ER⁺);
(ii) has at least one gain of function missense mutation within the ligand binding domain (LBD) of the Estrogen Receptor 1 (ESR1) gene; and
(iii) has increased expression of one or more genes other than the ESR1 gene.

25. A method of monitoring a patient on a breast cancer treatment, comprising:
(a) determining quantitative measures of the mutant allele frequency (MAF) in circulating tumor DNA (ctDNA) of at least one gain of function missense mutation within the ligand binding domain (LBD) of the Estrogen Receptor 1 (ESR1) gene (ESR1 ctDNA) in a biological sample of the patient, wherein the quantitative measures are performed over a period of time at determined intervals; and
(b) determining a positive predictive value (PPV) for clinical benefit with stable disease of the cancer treatment,
wherein the PPV indicates responsiveness to the cancer treatment, and
wherein the cancer treatment comprises an effective amount of lasofoxifene or a pharmaceutically acceptable salt thereof and an effective amount of a CDK4/6 inhibitor (CDK4/6i).

26. The method of claim 1, wherein progression of breast cancer in the patient is reduced.
